SEQUENCE LISTING (73 sequences)

SEQ ID NO: 1 - protein, 21 aa, Placobdella papillifera
  MOD_RES (6): Unknown amino acid
  MOD_RES (12): Unknown amino acid
  MOD_RES (19): Unknown amino acid
VEFQDXKKSSDXETLELRXNK

SEQ ID NO: 2 - protein, 8 aa, Placobdella papillifera
  MOD_RES (2): Unknown amino acid
LXWYGFTT

SEQ ID NO: 3 - protein, 20 aa, Placobdella papillifera
  MOD_RES (1): Unknown amino acid
  MOD_RES (5): Unknown amino acid
  MOD_RES (8): Unknown amino acid
  MOD_RES (10): Unknown amino acid
  MOD_RES (13): Unknown amino acid
  MOD_RES (18): Unknown amino acid
XEYGXKRXLXQGXNEAQXRK

SEQ ID NO: 4 - DNA, 286 bp, Placobdella papillifera
ccatcctaatacgactcactatagggctcgagcggccgcccgggcaggtatcataatgaagcaagtagccttattgttcatcatattggggtcggtagttttggcggtagagtttcaagattgcaagaaatcttcggattgtgaaactttggaattgaggtgcaacaaaaatacttccaaatgcgaatgtagaaatcaagtatgtcccagagcctgccccgatggtaaatacaaacttgatgaatacggctgcaagaggtgtctctgccaaggttgcaatgaagct

SEQ ID NO: 5 - DNA, 674 bp, Placobdella papillifera
gggtcggtagttttggcggtagagtttcaagattgcaagaaatcttcggattgtgaaactttggaattgaggtgcaacaaaaatacttccaaatgcgaatgtagaaatcaagtatgtcccagagcctgccccgatggtaaatacaaacttgatgaatacggctgcaagaggtgtctctgccaaggttgcaatgaagctcaatgtcgcaagttgtgttggtacggtttcaccacggatgaaaatggatgcgaatcttactgcaaatgcaacaccaaagaaacggcttgtaaaaacgttctgtgttcggattcttatcaatgcgatcctgaatcaggaaattgcgttgcagtgactcctggaaaagagcacgactattatagttataatgatgacgatgatgaagataaatgaaaacgaacgaacgactatataaaatatatcaattaaatatatttaacatatttaattattatattaaatatattaattattatattaaatgtaataaatatattaatcattaaataaatatattaaatatatttaattattatattatgtataataaattttattataaaaaaaaaaaaaaaaaaaaaaaaaaaaaagcggccgctgaattctaacctgcccgggcggccgctcgagccctatagtgagtcgtattaggatgg

SEQ ID NO: 6 - DNA, 444 bp, Placobdella papillifera
atgaagcaagtagccttattgttcatcatattggggtcggtagttttggcggtagagtttcaagattgcaagaaatcttcggattgtgaaactttggaattgaggtgcaacaaaaatacttccaaatgcgaatgtagaaatcaagtatgtcccagagcctgccccgatggtaaatacaaacttgatgaatacggctgcaagaggtgtctctgccaaggttgcaatgaagctcaatgtcgcaagttgtgttggtacggtttcaccacggatgaaaatggatgcgaatcttactgcaaatgcaacaccaaagaaacggcttgtaaaaacgttctgtgttcggattcttatcaatgcgatcctgaatcaggaaattgcgttgcagtgactcctggaaaagagcacgactattatagttataatgatgacgatgatgaagataaatga

SEQ ID NO: 7 - DNA, 475 bp, Placobdella papillifera
cgggcaggtatcctaatgaagcaagtagccttattgttcatcatattggggtcggtagttttggcagtagagtttcaagattgcaagaaatcttcggattgtgaaactttggaattaaggtgcaataaaaatacttccaaatgcgaatgtagaaatcaagtgtgtcccagagcctgccccgatggtaaatacaaacttgatgaatacggctgcaagaggtgtctctgccaaggttgcaatgaagctcaatgtcgcaagttgtgttggtacggtttcaccacggatgaaaatggatgcgaatcttactgcaaatgcaataccaaagaaacggcttgtaaaaacgttctgtgttcggattcttatcaatgcgatcctgaatcaggaaattgcgttgcagtgattcctggaaaagagcacgactattatagttataatgatgacgatgatgaagataaatgaaaacgaacgaacgact

SEQ ID NO: 8 - DNA, 472 bp, Placobdella papillifera
cgggcaggtatcataatgaagcaagtagctttattgtttatcatattggggtcggtagttttggcagtagagttccaagattgcaagaaatcttcggattgtgaaactttggaactgaggtgcaataaaaatacttccaaatgcgaatgtagaaatcaagtatgtcccagagcctgccccgatggtaaatacaaacttgatgaatacggctgcaagaggtgtctctgccaaggttgcaatgaagctcaatgtcgcaagttgtgttggtacggtttcactacggatgaaaatggatgcgaatcttactgcaaatgcaacaccaaagaaacggcctgtaaaaacgtactgtgttcggaatcataccaatgcgatcctgaatcaggaaattgcgttgcagtgattcctggaaaagagcacgactactatagttataatgatgatgatgaagataaatgaaaacgaacgaacgact

SEQ ID NO: 9 - DNA, 472 bp, Placobdella papillifera
cgggaaggtatcataatgaagcaagtagctttattgtttatcatattggggtcggtagttttggcagtagagttccaagattgcaagaaatcttcggattgtgaaactttggaactgaggtgcaataaaaatacttccaaatgcgaatgtagaaatcaagtatgtcccagagcctgccccgatggtaaatacaaacttgatgaatacggctgcaagaggtgtctctgccaaggttgcaatgaagctcaatgtcgcaagttgtgttggtacggtttcactacggatgaaaatggatgcgaatcttactgcaaatgcaacaccaaagaaacggcctgtaaaaacgtactgtgttcggaatcataccaatgcgatcctgaatcaggaaattgcgttgcagtgattcctggaaaagagcacgactactatagttataatgatgatgatgaagataaatgaaaacgaacgaacgact

SEQ ID NO: 10 - protein, 147 aa, Placobdella papillifera
MKQVALLFIILGSVVLAVEFQDCKKSSDCETLELRCNKNTSKCECRNQVCPRACPDGKYKLDEYGCKRCLCQGCNEAQCRKLCWYGFTTDENGCESYCKCNTKETACKNVLCSDSYQCDPESGNCVAVTPGKEHDYYSYNDDDDEDK

SEQ ID NO: 11 - protein, 147 aa, Placobdella papillifera
MKQVALLFIILGSVVLAVEFQDCKKSSDCETLELRCNKNTSKCECRNQVCPRACPDGKYKLDEYGCKRCLCQGCNEAQCRKLCWYGFTTDENGCESYCKCNTKETACKNVLCSDSYQCDPESGNCVAVIPGKEHDYYSYNDDDDEDK

SEQ ID NO: 12 - protein, 146 aa, Placobdella papillifera
MKQVALLFIILGSVVLAVEFQDCKKSSDCETLELRCNKNTSKCECRNQVCPRACPDGKYKLDEYGCKRCLCQGCNEAQCRKLCWYGFTTDENGCESYCKCNTKETACKNVLCSESYQCDPESGNCVAVIPGKEHDYYSYNDDDEDK

SEQ ID NO: 13 - DNA, 715 bp, Placobdella papillifera
gcccaccatcgggcgcggatcccggtccgaagcgcgcggaattctaaatatgaaattcttagtcaacgttgcccttgtttttatggtcgtatacatttcttacatctatgcggtagagtttcaagattgcaagaaatcttcggattgtgaaactttggaattaaggtgcaataaaaatacttccaaatgcgaatgtagaaatcaagtgtgtcccagagcctgccccgatggtaaatacaaacttgatgaatacggctgcaagaggtgtctctgccaaggttgcaatgaagctcaatgtcgcaagttgtgttggtacggtttcaccacggatgaaaatggatgcgaatcttactgcaaatgcaataccaaagaaacggcttgtaaaaacgttctgtgttcggattcttatcaatgcgatcctgaatcaggaaattgcgttgcagtgattcctggaaaagagcacgactattatagttataatgatgacgatgatgaagataaatgaaaacgaacgaacgactctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagca

SEQ ID NO: 14 - protein, 63 aa, Placobdella papillifera
VEFQDCKKSSDCETLELRCNKNTSKCECRNQVCPRACPDGKYKLDEYGCKRCLCQGCNEAQCR

SEQ ID NO: 15 - protein, 130 aa, Placobdella papillifera
VEFQDCKKSSDCETLELRCNKNTSKCECRNQVCPRACPDGKYKLDEYGCKRCLCQGCNEAQCRKLCWYGFTTDENGCESYCKCNTKETACKNVLCSDSYQCDPESGNCVAVTPGKEHDYYSYNDDDDEDK

SEQ ID NO: 16 - protein, 130 aa, Placobdella papillifera
VEFQDCKKSSDCETLELRCNKNTSKCECRNQVCPRACPDGKYKLDEYGCKRCLCQGCNEAQCRKLCWYGFTTDENGCESYCKCNTKETACKNVLCSDSYQCDPESGNCVAVIPGKEHDYYSYNDDDDEDK

SEQ ID NO: 17 - protein, 129 aa, Placobdella papillifera
VEFQDCKKSSDCETLELRCNKNTSKCECRNQVCPRACPDGKYKLDEYGCKRCLCQGCNEAQCRKLCWYGFTTDENGCESYCKCNTKETACKNVLCSESYQCDPESGNCVAVIPGKEHDYYSYNDDDEDK

SEQ ID NO: 18 - protein, 75 aa, Placobdella papillifera
GCNEAQCRKLCWYGFTTDENGCESYCKCNTKETACKNVLCSDSYQCDPESGNCVAVTPGKEHDYYSYNDDDDEDK

SEQ ID NO: 19 - protein, 75 aa, Placobdella papillifera
GCNEAQCRKLCWYGFTTDENGCESYCKCNTKETACKNVLCSDSYQCDPESGNCVAVIPGKEHDYYSYNDDDDEDK

SEQ ID NO: 20 - protein, 74 aa, Placobdella papillifera
GCNEAQCRKLCWYGFTTDENGCESYCKCNTKETACKNVLCSESYQCDPESGNCVAVIPGKEHDYYSYNDDDEDK

SEQ ID NO: 21 - protein, 76 aa, Placobdella papillifera
QGCNEAQCRKLCWYGFTTDENGCESYCKCNTKETACKNVLCSDSYQCDPESGNCVAVTPGKEHDYYSYNDDDDEDK

SEQ ID NO: 22 - protein, 76 aa, Placobdella papillifera
QGCNEAQCRKLCWYGFTTDENGCESYCKCNTKETACKNVLCSDSYQCDPESGNCVAVIPGKEHDYYSYNDDDDEDK

SEQ ID NO: 23 - protein, 75 aa, Placobdella papillifera
QGCNEAQCRKLCWYGFTTDENGCESYCKCNTKETACKNVLCSESYQCDPESGNCVAVIPGKEHDYYSYNDDDEDK

SEQ ID NO: 24 - protein, 104 aa, Placobdella papillifera
ECRNQVCPRACPDGKYKLDEYGCKRCLCQGCNEAQCRKLCWYGFTTDENGCESYCKCNTKETACKNVLCSDSYQCDPESGNCVAVTPGKEHDYYSYNDDDDEDK

SEQ ID NO: 25 - protein, 104 aa, Placobdella papillifera
ECRNQVCPRACPDGKYKLDEYGCKRCLCQGCNEAQCRKLCWYGFTTDENGCESYCKCNTKETACKNVLCSDSYQCDPESGNCVAVIPGKEHDYYSYNDDDDEDK

SEQ ID NO: 26 - protein, 103 aa, Placobdella papillifera
ECRNQVCPRACPDGKYKLDEYGCKRCLCQGCNEAQCRKLCWYGFTTDENGCESYCKCNTKETACKNVLCSESYQCDPESGNCVAVIPGKEHDYYSYNDDDEDK

SEQ ID NO: 27-29 - skipped (3 entries)

SEQ ID NO: 30 - protein, 67 aa, Placobdella papillifera
KLCWYGFTTDENGCESYCKCNTKETACKNVLCSDSYQCDPESGNCVAVTPGKEHDYYSYNDDDDEDK

SEQ ID NO: 31 - protein, 67 aa, Placobdella papillifera
KLCWYGFTTDENGCESYCKCNTKETACKNVLCSDSYQCDPESGNCVAVIPGKEHDYYSYNDDDDEDK

SEQ ID NO: 32 - protein, 66 aa, Placobdella papillifera
KLCWYGFTTDENGCESYCKCNTKETACKNVLCSESYQCDPESGNCVAVIPGKEHDYYSYNDDDEDK

SEQ ID NO: 33 - DNA, 20 bp, Artificial Sequence
  Description of Artificial Sequence: Primer
  modified_base (12): a, t, c or g
gcytcrttrcanccytgrca

SEQ ID NO: 34 - DNA, 25 bp, Artificial Sequence
  Description of Artificial Sequence: Primer
ggggtcggtagttttggcggtagag

SEQ ID NO: 35 - DNA, 18 bp, Artificial Sequence
  Description of Artificial Sequence: Primer
cgggcaggtatcataatg

SEQ ID NO: 36 - DNA, 18 bp, Artificial Sequence
  Description of Artificial Sequence: Primer
agtcgttcgttcgttttc

SEQ ID NO: 37 - DNA, 32 bp, Artificial Sequence
  Description of Artificial Sequence: Synthetic oligonucleotide
actgcagagtcgttcgttcgttttcatttatc

SEQ ID NO: 38 - protein, 17 aa, Placobdella papillifera
MKQVALLFIILGSVVLA

SEQ ID NO: 39 - protein, 21 aa, Placobdella papillifera
MKFLVNVALVFMVVYISYIYA

SEQ ID NO: 40 - DNA, 96 bp, Artificial Sequence
  Description of Artificial Sequence: Synthetic oligonucleotide
agaattctaaatatgaaattcttagtcaacgttgcccttgtttttatggtcgtatacatttcttacatctatgcggtagagtttcaagattgcaag

SEQ ID NO: 41-49 - skipped (9 entries)

SEQ ID NO: 50 - protein, 22 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
  MOD_RES (22): Any amino acid
  See specification as filed for detailed description of substitutions and preferred embodiments
XEFQDXKKSSDXETLELRXNKX

SEQ ID NO: 51 - protein, 34 aa, Artificial Sequence
  Description of Artificial Sequence: Formula sequence
  MOD_RES (34): Any amino acid
  See specification as filed for detailed description of substitutions and preferred embodiments
NTSKCECRNQVCPRACPDGKYKLDEYGCKRCLCX

SEQ ID NO: 52-53 - skipped (2 entries)

SEQ ID NO: 54 - protein, 9 aa, Placobdella papillifera
QGCNEAQCR

SEQ ID NO: 55-59 - skipped (5 entries)

SEQ ID NO: 60 - protein, 68 aa, Artificial Sequence
  Description of Artificial Sequence: Formula sequence
  MOD_RES (1): Any amino acid or not present
  MOD_RES (35): Asp or Glu
  MOD_RES (50): Thr or Ile
  MOD_RES (65)..(66): Asp or Glu
  MOD_RES (67): Asp or Lys
  MOD_RES (68): Lys or not present
  See specification as filed for detailed description of substitutions and preferred embodiments
XKLCWYGFTTDENGCESYCKCNTKETACKNVLCSXSYQCDPESGNCVAVXPGKEHDYYSYNDDDXXXX

SEQ ID NO: 61 - protein, 63 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
  residues 1-56 may or may not be present; if residues 1-56 do not embody SEQ ID NO: 62, then Gly or Gln-Gly may encompass this region
  See specification as filed for detailed description of substitutions and preferred embodiments
XEFQDXKKSSDXETLELRXNKNTSKCECRNQVCPRACPDGKYKLDEYGCKRCLCQGCNEAQCR

SEQ ID NO: 62 - protein, 56 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
  residues 1-26 may or may not be present
  See specification as filed for detailed description of substitutions and preferred embodiments
XEFQDXKKSSDXETLELRXNKNTSKCECRNQVCPRACPDGKYKLDEYGCKRCLCQG

SEQ ID NO: 63 - protein, 26 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
  See specification as filed for detailed description of substitutions and preferred embodiments
XEFQDXKKSSDXETLELRXNKNTSKC

SEQ ID NO: 64 - protein, 22 aa, Artificial Sequence
  Description of Artificial Sequence: Formula sequence
  MOD_RES (6): Any amino acid
  MOD_RES (12): Any amino acid
  MOD_RES (19): Any amino acid
  MOD_RES (22): Any amino acid
  See specification as filed for detailed description of substitutions and preferred embodiments
VEFQDXKKSSDXETLELRXNKX

SEQ ID NO: 65 - protein, 55 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
  MOD_RES (55): Any amino acid
  See specification as filed for detailed description of substitutions and preferred embodiments
XEFQDXKKSSDXETLELRXNKNTSKCECRNQVCPRACPDGKYKLDEYGCKRCLCX

SEQ ID NO: 66 - protein, 63 aa, Artificial Sequence; identical to SEQ ID NO: 61
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid

SEQ ID NO: 67 - protein, 131 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
XEFQDXKKSSDXETLELRXNKNTSKCECRNQVCPRACPDGKYKLDEYGCKRCLCQGCNEAQCRKLCWYGFTTDENGCESYCKCNTKETACKNVLCSDSYQCDPESGNCVAVTPGKEHDYYSYNDDDDEEDK

SEQ ID NO: 68 - protein, 130 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
XEFQDXKKSSDXETLELRXNKNTSKCECRNQVCPRACPDGKYKLDEYGCKRCLCQGCNEAQCRKLCWYGFTTDENGCESYCKCNTKETACKNVLCSDSYQCDPESGNCVAVIPGKEHDYYSYNDDDDEDK

SEQ ID NO: 69 - protein, 129 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
XEFQDXKKSSDXETLELRXNKNTSKCECRNQVCPRACPDGKYKLDEYGCKRCLCQGCNEAQCRKLCWYGFTTDENGCESYCKCNTKETACKNVLCSESYQCDPESGNCVAVIPGKEHDYYSYNDDDEDK

SEQ ID NO: 70 - protein, 68 aa, Artificial Sequence
  Description of Artificial Sequence: Formula sequence
  MOD_RES (1): Any amino acid or not present
  See specification as filed for detailed description of substitutions and preferred embodiments
XKLCWYGFTTDENGCESYCKCNTKETACKNVLCSDSYQCDPESGNCVAVTPGKEHDYYSYNDDDDEDK

SEQ ID NO: 71 - protein, 68 aa, Artificial Sequence
  Description of Artificial Sequence: Formula sequence
  MOD_RES (1): Any amino acid or not present
  See specification as filed for detailed description of substitutions and preferred embodiments
XKLCWYGFTTDENGCESYCKCNTKETACKNVLCSDSYQCDPESGNCVAVIPGKEHDYYSYNDDDDEDK

SEQ ID NO: 72 - protein, 67 aa, Artificial Sequence
  Description of Artificial Sequence: Formula sequence
  MOD_RES (1): Any amino acid or not present
  See specification as filed for detailed description of substitutions and preferred embodiments
XKLCWYGFTTDENGCESYCKCNTKETACKNVLCSESYQCDPESGNCVAVIPGKEHDYYSYNDDDEDK

SEQ ID NO: 73 - protein, 130 aa, Artificial Sequence
  Description of Artificial Sequence: Formula sequence
  MOD_RES (1): Any amino acid or not present
  MOD_RES (6): Any amino acid
  MOD_RES (12): Any amino acid
  MOD_RES (19): Any amino acid
  MOD_RES (97): Any amino acid
  MOD_RES (112): Any amino acid
  MOD_RES (127)..(128): Asp or Glu
  MOD_RES (129): Asp or Lys
  MOD_RES (130): Lys or not present
  residues 1-56 may or may not be present; if residues 1-56 do not embody SEQ ID NO: 62, then Gly or Gln-Gly may encompass this region
  See specification as filed for detailed description of substitutions and preferred embodiments
XEFQDXKKSSDXETLELRXNKNTSKCECRNQVCPRACPDGKYKLDEYGCKRCLCQGCNEAQCRKLCWYGFTTDENGCESYCKCNTKETACKNVLCSXSYQCDPESGNCVAVXPGKEHDYYSYNDDDXXXX

(19) United States
(12) Patent Application Publication
(10) Pub. No.: US 2004/0038869 A1
(43) Pub. Date: Feb. 26, 2004

(54) Inhibitors of complement activation, their preparation and use

(76) Inventors: Sarah Finney, Swansea (GB); Lisa Seale, Swansea (GB); Robert Brian Wallis, Swansea (GB)

Correspondence Address: YOUNG & THOMPSON, 745 SOUTH 23RD STREET 2ND FLOOR, ARLINGTON, VA, 22202

(21) Appl. No.: 10/168,948

(22) PCT Filed: Dec. 21, 2000
(86) PCT No.: PCT/GB00/04971

(30) Foreign Application Priority Data
Dec. 24, 1999 (GB) 9930659.9

(51) Int. Cl. A61K 38/17; A61K 38/14; C07K 9/00

(52) U.S. Cl. 514/8; 514/12; 530/322; 530/324

(57) ABSTRACT

Polypeptides are disclosed that are capable of being isolated from ectoparasitic leeches which inhibit the alternative route of complement activation but which have substantially no effect on complement activation by the classical route. In one embodiment of the invention, the polypeptides have the following general formula [SEQ ID NO: 50] in which amino acids are represented by their conventional single letter codes: X1-E-F-Q-D-X2-K-K-S-S-D-X3-E-T-L-E-L-R-X4-N-K-X5, wherein: X1 is a hydrogen atom (H) or any naturally-occurring amino acid, preferably valine, or a sequence of amino acids; X2 is any single amino acid, preferably cysteine; X3 is any single amino acid, preferably cysteine; X4 is any single amino acid, preferably cysteine; X5 is an amino acid sequence comprising naturally-occurring amino acids, one or more of which may comprise post-translational modifications, such as glycosylation at asparagine, serine or threonine; and/or sulphato- or phospho- groups on tyrosine, such as are commonly found in polypeptides derived from leeches. The polypeptides can be prepared from leech species of the order Rhynchobdellida and more particularly those of the genus Placobdella, especially of the species Placobdella papillifera. Alternatively, the polypeptides can be synthesised chemically or produced by transgenic organisms carrying DNA sequences which encode them. Accordingly, also disclosed are nucleic acid sequences capable of expressing the polypeptides; hosts, and vectors comprising these sequences; and the use of the nucleic acids and polypeptides in therapy.

The present invention is concerned with a novel class of inhibitors of the alternative complement pathway that can be derived, for example, from mammalian parasite tissues or secretions, or can be produced by cultivation of appropriately genetically-modified organisms.

The complement system is a group of plasma proteins, the main physiological function of which is to mediate inflammation and to eliminate foreign organisms. It forms a complex enzyme cascade which, when activated, leads to the production of the chemotactic and vasoactive anaphylatoxins, C3a and C5a; the opsonin, C3b, which coats invading organisms causing recognition by phagocytic cells (so-called immune adherence; opsonins are proteins that coat a cell); and the cytolytic “membrane attack complex”, which lyses target cells directly. It causes a localised inflammatory response resulting in changes in vascular permeability, vasoconstriction, leucocyte activation and migration, and smooth muscle contraction.

Like all physiological mechanisms, complement can be activated inappropriately and, in such circumstances, causes marked inflammation. Complement is involved in the pathology of a large number of inflammatory and auto-immune diseases. It is therefore desirable to provide new inhibitors of the complement pathway for the treatment of such diseases; to reduce tissue rejection of implanted organs; and to inhibit complement activation on foreign surfaces, such as haemodialysers and cardio-pulmonary bypass circuits.

Activation of complement can occur through one or both of two pathways: the classical pathway and the alternative pathway, which merge together at the activation of a protein called C5 and then follow a common route.

The first part of the classical complement pathway is analogous to the blood coagulation cascade, another complex enzyme cascade, but which leads to the clotting place on an antibody-coated surface or other negatively-charged surface, and gives rise to a serine protease, C1 esterase. This, in the presence of an activated cofactor (C4b), specifically cleaves the next zymogen (C2) in the cascade (zymogens are pro-enzymes for serine proteases). The cascade leads eventually to an end-product which, in this case, is the membrane attack complex that lyses the target cell. The classical pathway also leads to the production of various by-products, which are themselves biologically active and act to potentiate the inflammatory response.

For the alternative pathway to become activated, the stimulus is normally provided by bacterial lipopolysaccharides; yeast (zymosan) or plant (inulin) polysaccharides; various polyanions, such as dextran sulphate, or the antibody-binding portions of immunoglobulins, including IgA and IgE. In addition, the alternative pathway seems to be activated by negatively-charged phospholipids. Normally, these phospholipids occur only on the intracellular face of cell membranes, but during trauma or, as in the case of platelets, activation, they migrate to the outer surface. All of these surfaces can bind any C3b, which circulates at low concentration, even in healthy individuals. C3b, in turn (as shown in the flow chart below), binds circulating factor B, which is then presented in a form that can be cleaved by factor D, a circulating serine protease. The resulting C3bBb complex is a potent C3 convertase, which cleaves C3 into two fragments, C3a and C3b. C3a has potent pro-inflammatory effects. C3b becomes available not only for complexation to more surfaces and factor B causing acceleration of activation, but also for binding to the C3bBb complex to form C5 convertase. Any C3b formed in the initiation phase of activation in excess of that required for complexation with Bb will coat foreign particles, allowing recognition by phagocytic cells. This increased affinity of phagocytic cells for C3b-coated particles is known as “immune adherence”.

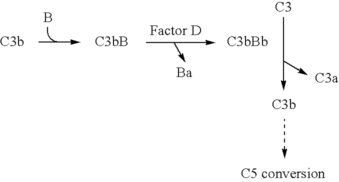

Activation of complement by the classical pathway is a high throughput mechanism that is usually initiated by antibodies binding to antigens. In order for this to provide sufficient activation to eliminate, for example, infection, a large number of antibody-antigen complexes need to be formed. This is normally only associated with late-stage activation, that is when specific antibodies to the foreign body have been generated. Conversely, activation of the alternative pathway can occur at very low concentrations of antibodies and probably occurs spontaneously at a low rate, which is under the control of a variety of inhibitors. Therefore, it is probably the first complement pathway to become activated since, unlike the classical route, it does not require the involvement of large amounts of specific antibodies. In normal circumstances, the physiological rôle of complement activation is to clear infections by removing invading organisms. However, like all physiological mechanisms, it can happen inappropriately and lead to pathologies that are associated with morbidity and mortality. Some of the pathologies can arise through a deficiency of one of the control proteins. For example, C1 inhibitor deficiency causes an inflammatory disease known as hereditary angioneurotic oedema. Other problems are caused either by antibodies directed against self (for example, auto-immune diseases such as rheumatoid arthritis); or by complement activation on damaged tissue or cells (for example, sickle cell anaemia), or on foreign surfaces (for example haemodialysers). Such conditions are usually treated to alleviate symptoms only or are left untreated. There is therefore a need for means to inhibit complement activation and consequently modify the cause of the disease.

The activation of blood enzyme systems by contact with foreign surfaces is well documented. Not only is the intrinsic pathway of blood coagulation activated, but also activation of complement takes place. Cellulosic haemodialysis membranes lead to increased concentrations of the degradation products of both C3 and C4, indicating activation of both pathways [Innes A, Farrell A M, Burden R P, Morgan A G, Powell R J. J Clin Pathol 47: 155-158 (1994)] although the alternative pathway seems to dominate. Complement activation has been presumed to be responsible for-neutrophil activation leading to protcase secretion, neutropenia and pulmonary artery hypertension, all side effects of this procedure. Activation of the alternative pathway is also well known in cardio-pulmonary bypass and is thought to be an important cause of neutrophil stimulation, lung injury and endothelial dysfunction, none of which occur in dogs which are deficient in this pathway [Finn A, Morgan B P, Rebuck N, Klein N, Rogers C A, Hibbs M, Elliott M, Shore D F, Evans T W, Strobel S, Moat N. J Thoracic Cardiovasc Surg 111: 451-459 (1996)].

Another instance where the immune system becomes inappropriately activated is in rejection of transplanted tissue. There is a shortage of human donor organs that could be relieved with the use of organs from other species (so-called xenotransplantation) if the problems of hyperacute rejection could be overcome. One of the major mediators of hyperacute rejection is complement, and its activation through both the alternative and classical pathway leads to endothelial cell activation, thrombosis, intravascular coagulation, oedema and eventual loss of function of the transplanted organ. A particular problem is microcirculatory occlusion by accumulating polymorphonuclear leukocytes. Whilst it is unlikely that complement inhibitors will be the total solution to these problems, there is good evidence that they will provide significant benefit and, perhaps in combination with other immunosuppressants, will provide enabling therapy.

Complement is known to become activated in auto-immune disease. In rheumatoid arthritis, marked increases in C1 inhibitor-C1 complexes and in C3a have been shown. The increase in the complement activation products correlate with disease severity scores and with diagnostic markers of neutrophil activation such as lactoferrin, thus inhibitors of complement can be expected to relieve the complement-mediated symptoms of this and other auto-immune diseases. Complement activation is well documented in sepsis in humans, since the products of activation (namely C3a, C3d, C5a and C4) are elevated, and correlate with severity of the disease and with fatal outcome. In one small study of five patients, purified C1 inhibitor has been administered and was associated with attenuation of complement activation and improvement in symptoms [Hack C E, Voerman H J, Eisele B, Keinecke H-O, Nuijens J H, Eerenberg A J M, Ogilvie A, van Schijndel R J M S, Delvos U, Thijs L G. Lancet 339: 378 (1992)]. Elevated products of complement activation have also been reported in asthma, systemic lupus erythematosis, Alzheimer's disease and sickle cell disease.

Despite being implicated in a wide range of debilitating conditions, therapeutic agents that inhibit complement are, as yet, unavailable for human use. Ai inhibitor in tick saliva of the alternative complement pathway, and particularly of C3b deposition to activating surfaces, has previously been described [Ribeiro J M C. Exp Parasitol 64: 347-353 (1987)]. Genetically-engineered soluble human complement receptor 1 (sCR1) has been shown to reduce kidney damage in an animal model of glomerulonephritis [Couser W G, Johnson R J, Young B A, Yeh G, Toth C A, Rudolph A R. J Am Soc Nephrology 5: 1888-1894 (1995)]. Intra-articular injection in adjuvant-induced arthritic rats caused a reduction in joint swelling and synovitis [Goodfellow R M, Williams A S, Levin J L, Williams B D, Morgan B P. Clin Exp Immunol 110: 45-52 (1997)]. Such inhibitors could potentially be of great use in many other diseases where complement is implicated.

With no effective therapies available for the treatment or prevention of many inflammatory and auto-immune diseases, it is a purpose of one aspect of the present invention to provide an inhibitor of the alternative complement pathway that is suitable for pharmaceutical or therapeutic use. To this end, we provide new polypeptides capable of being isolated from ectoparasitic leeches, which polypeptides inhibit the alternative route of complement activation but which have no effect on complement activation by the classical route.

In one embodiment of the invention, the polypeptides have the following general formula [SEQ ID NO: 50] in which amino acids are represented by their conventional single letter codes:

X1-E-F-Q-D-X2-K-K-S-S-D-X3-E-T-L-E-L-R-X4-N-K-X5 [SEQ ID NO: 50]

wherein:

X1 is a hydrogen atom (H) or any naturally-occurring amino acid, preferably valine, or a sequence of amino acids;

X2 is any single amino acid, preferably cysteine;

X3 is any single amino acid, preferably cysteine;

X4 is any single amino acid, preferably cysteine;

X5 is an amino acid sequence comprising naturally-occurring amino acids, one or more of which may comprise post-translational modifications, such as glycosylation at asparagine, serine or threonine; and/or sulphato- or phospho- groups on tyrosine, such as are commonly found in polypeptides derived from leeches.

When X1 is valine, the polypeptides of the invention are of the above general formula [SEQ ID NO: 50] in which the first 21 amino acids from the N-terminus of the mature polypeptide are of [SEQ ID NO: 1]:

V-E-F-Q-D-X-K-K-S-S-D-X-E-T-L-E-L-R-X-N-K- [SEQ ID NO: 1]

Still more preferred are polypeptides wherein each of X, X2, X3 and X4 are cysteine in the above formulae.

In particular, the polypeptides can have the above general formula [SEQ ID NO: 50] wherein X5 is the amino acid sequence [SEQ ID NO: 51]:

-N-T-S-K-C-E-C-R-N-Q-V-C-P-R-A-C-P-D-G-K-Y-K-L-D-E-Y-G-C-K-R-C-L-C-X6 [SEQ ID NO: 51]

wherein X6 is an amino acid sequence comprising naturally-occurring amino acids, one or more of which may comprise post-translational modifications, such as glycosylation at asparagine, serine or threonine; and/or sulphato- or phospho- groups on tyrosine, such as are commonly found in polypeptides derived from leeches.

More particularly, the polypeptides can comprise the above formula [SEQ ID NO: 51] wherein X6 is an amino acid sequence selected from one of the following [SEQ ID NOS: 54 and 21 to 23]:

-Q-G-C-N-E-A-Q-C-R [SEQ ID NO: 54];

[SEQ ID NO: 54]

-Q-G-C-N-E-A-Q-C-R;

[SEQ ID NO: 21]

-Q-G-C-N-E-A-Q-C-R-K-L-C-W-Y-G-F-T-T-D-E-N-G-
C-E-S-Y-C-K-C-N-T-K-E-T-A-C-K-N-V-L-C-S-D-S-Y-
Q-C-D-P-E-S-G-N-C-V-A-V-T-P-G-K-E-H-D-Y-Y-S-Y
N-D-D-D-D-E-D-K;

[SEQ ID NO: 22]

-Q-G-C-N-E-A-Q-C-R-K-L-C-W-Y-G-F-T-T-D-E-N-G-C
-E-S-Y-C-K-C-N-T-K-.E-T-A-C-K-N-V-L-C-S-D-S-Y-Q
-C-D-P-E-S-G-N-C-V-A-V-I-P-G-K-E-H-D-Y-Y-S-Y-N
D-D-D-D-E-D-K:
and

[SEQ ID NO: 23]

-Q-G-C-N-E-A-Q-C-R-K-L-C-W-Y-G-F-T-T-D-E-N-G-
C-E-S-Y-C-K-C-N-T-K-E-T-A-C-K-N-V-L-C-S-E-S-Y-
Q-C-D-P-E-S-G-N-C-V-A-V-I-P-G-K-E-H-D-Y-Y-S-Y
N-D-D-D-E-D-K.

In a second embodiment of the invention, the polypeptides have the following general formula [SEQ ID NO: 60]:

[SEQ ID NO: 60]

X7-K-L-C-W-Y-G-F-T-T-D-E-N-G-C-E-S-Y-C-K-C-N-T
-K-B-T-A-C-K-N-V-L-C-S-X8-S-Y-Q-C-D-P-E-S-G-N-C
-V-A-V-X9-P-G-K-E-H-D-Y-Y-S-Y-N-D-D-D-X10

wherein:

X7 is either a hydrogen atom or an amino acid sequence comprising naturally-occurring amino acids, one or more of which may have post-translational modifications such as glycosylation at asparagine, serine or threonine;

X8 is D or E;

X9 is T or I; and

X10 is -D-E-D-K or -E-D-K

Preferably, in [SEQ ID NO: 60], X7 is bonded to a peptide in which:

X8 is D, X9 is T and X10 is -D-E-D-K, as in [SEQ ID NO: 30]:

| | |
|---|---|
| X8 is D, X9 is T and X10 is -D-E-D-K, as in [SEQ ID NO: 30] | |
| -K-L-C-W-Y-G-F-T-T-D-E-N-G-C-E-S-Y-C-K-C-N-T-K-E-T-A-C-K-N-V-L-C-S-D- | [SEQ ID NO: 30] |
| S-Y-Q-C-D-P-E-S-G-N-C-V-A-V-T-P-G-K-E-H-D-Y-Y-S-Y-N-D-D-D-D-E-D-K | |
| or | |
| X8 is D, X9 is 1 and X10 is -D-E-D-K, as in [SEQ ID NO: 31]: | |
| -K-L-C-W-Y-G-F-T-T-D-E-N-G-C-E-S.Y-C-K-C-N-T-K-E-T-A-C-K-N-V-L-C-S-D- | [SEQ ID NO: 31] |
| S-Y-Q-C-D-P-E-S-G-N-C-V-A-V-I-P-G-K-E-H-D-Y-Y-S-Y-N-D-D-D-D-E-D-K | |
| or | |
| X8 is E, X9 is 1 and X10 is -E-D-K, as in [SEQ ID NO:32]: | |
| -K-L-C-W-Y-G-F-T-T-D-E-N-G-C-E-S-Y-C-K-C-N-T-K-E-T-A-C-K-N-V-L-C-S-E- | [SEQ ID NO: 32] |
| S-Y-Q-C-D-P-E-S-G-N-C-V-A-V-I-P-G-K-E-H-D-Y-Y-S-Y-N-D-D-D-E-D-K | |

Preferably, X7 is the sequence [SEQ ID NO: 61]:

X11-C-N-E-A-Q-C-R- [SEQ ID NO: 61]

wherein X11 is selected from G-; Q-G-; and [SEQ ID NO: 62]:

X12-E-C-R-N-Q-V-C-P-R-A-C-P-D-G-K-Y-K-L-D-E-Y-G-C-K-R-C-L-C-Q-G- [SEQ ID NO: 62]

wherein X12 is either a hydrogen atom or [SEQ ID NO: 63]:

X13-N-T-S-K-C- [SEQ ID NO: 63],

wherein X13 is a sequence of [SEQ ID NQ: 50].

When X12 is a hydrogen atom then, more preferably:

when X7 is bonded to a peptide of [SEQ ID NO: 30] as defined above, then X11 is G (ie [SEQ ID NO: 18]); Q-G (ie [SEQ ID NO: 21]); or [SEQ ID NO: 62] as defined above (ie [SEQ ID NO: 24]), that is:

| | |
|---|---|
| G-C-N-E-A-Q-C-R-K-L-C-W-Y-G-F-T-T-D-E-N-G-C-E-S-Y-C-K-C-N-T-K-E-T-A- | [SEQ ID NO: 18] |
| C-K-N-V-L-C-S-D-S-Y-Q-C-D-P-E-S-G-N-C-V-A-V-T-P-G-K-E-H-D-Y-Y-S-Y-N- | |
| D-D-D-D-E-D-K; | |

| | |
|---|---|
| Q-G-C-N-E-A-Q-C-R-K-L-C-W-Y-G-F-T-T-D-E-N-G-C-E-S-Y-C-K-C-N-T-K-E-T- | [SEQ ID NO: 21] |
| A-C-K-N-V-L-C-S-D-S-Y-Q-C-D-P-E-S-G-N-C-V-A-V-T-P-G-K-E-H-D-Y-Y-S-Y | |
| N-D-D-D-D-E-D-K; | |
| or | |
| E-C-R-N-Q-V-C-P-R-A-C-P-D-G-K-Y-K-L-D-E-Y-G-C-K-R-C-L-C-Q-G-C-N-E-A- | [SEQ ID NO: 24] |
| Q-C-R-K-L-C-W-Y-G-F-T-T-D-E-N-G-C-E-S-Y-C-K-C-N-T-K-E-T-A-C-K-N-V-L- | |
| C-S-D-S-Y-Q-C-D-P-E-S-G-N-C-V-A-V-T-P-G-K-E-H-D-Y-Y-S-Y-N-D-D-D-D-E- | |
| D-K; | |

when X7 is bonded to a peptide of [SEQ ID NO: 31] as defined above, then X11 is G (ie [SEQ ID NO: 19]); Q-G (ie [SEQ ID NO: 22]); or [SEQ ID NO: 62] as defined above (ie [SEQ ID NO: 25]; that is:

| | |
|---|---|
| G-C-N-E-A-Q-C-R-K-L-C-W-Y-G-F-T-T-D-E-N-G-C-E-S-Y-C-K-C-N-T-K-E-T-A- | [SEQ ID NO: 19] |
| C-K-N-V-L-C-S-D-S-Y-Q-C-D-P-E-S-G-N-C-V-A-V-I-P-G-K-E-H-D-Y-Y-S-Y-N- | |
| D-D-D-D-E-D-K; | |

| | |
|---|---|
| Q-G-C-N-E-A-Q-C-R-K-L-C-W-Y-G-F-T-T-D-E-N-G-C-E-S-Y-C-K-C-N-T-K-E-T- | [SEQ ID NO: 22] |
| A-C-K-N-V-L-C-S-D-S-Y-Q-C-D-P-E-S-G-N-C-V-A-V-I-P-G-K-E-H-D-Y-Y-S-Y- | |
| N-D-D-D-D-E-D-K; | |
| or | |
| E-C-R-N-Q-V-C-P-R-A-C-P-D-G-K-Y-K-L-D-E-Y-G-C-K-R-C-L-C-Q-G-C-N-E-A- | [SEQ ID NO: 25] |
| Q-C-R-K-L-C-W-Y-G-F-T-T-D-E-N-G-C-E-S-Y-C-K-C-N-T-K-E-T-A-C-K-N-V | |
| L-C-S-D-S-Y-Q-C-D-P-E-S-G-N-C-V-A-V-I-P-G-K-E-H-D-Y-Y-S-Y-D-D-E-D-K; | |

and,

when X7 is bonded to a peptide of [SEQ ID NO: 32] as defined above, then X11 is G (ie [SEQ ID NO: 20]); Q-G (ie [SEQ ID NO: 23]); or [SEQ ID NO: 62] as defined above (ie [SEQ ID NO: 26]), that is:

| | |
|---|---|
| G-C-N-E-A-Q-C-R-K-L-C-W-Y-G-F-T-T-D-E-N-G-C-E-S-Y-C-K-C-N-T-K-E-T-A- | [SEQ ID NO: 20] |
| C-K-N-V-L-C-S-E-S-Y-Q-C-D-P-E-S-G-N-C-V-A-V-P-G-K-E-H-D-Y-Y-S-Y-N- | |
| D-D-D-E-D-K; | |

| | |
|---|---|
| Q-G-C-N-E-A-Q-C-R-K-L-C-W-Y-G-F-T-T-D-E-N-G-C-E-S-Y-C-K-C-N-T-K-E-T- | [SEQ ID NO: 23] |
| A-C-K-N-V-L-C-S-E-S-Y-Q-C-D-P-E-S-G-N-C-V-A-V-I-P-G-K-E-H-D-Y-Y-S-Y- | |
| N-D-D-D-E-D-K; | |
| or | |
| E-C-R-N-Q-V-C-P-R-A-C-P-D-G-K-Y-K-L-D-E-Y-G-C-K-R-C-L-C-Q-G-C-N-E-A- | [SEQ ID NO:26] |
| Q-C-R-K-L-C-W-Y-G-F-T-T-D-E-N-G-C-E-S-Y-C-K-C-N-T-K-T-A-C-K-N-V-L- | |
| C-S-E-S-Y-Q-C-D-P-E-S-G-N-C-V-A-V-I-P-G-K-E-H-D-Y-Y-S-Y-N-D-D-D-E-D- | |
| K. | |

The polypeptides of this invention further include derivatives of those described hereinbefore having substantially similar or the same biological activity thereas. By ‘derivatives’ in this context are included bioprecursors, such as sequences as described hereinbefore further comprising a leader or signal sequence as described further herinafter; modifications thereof, such as sequences modified (eg glycosylated, or sulphated or phosphated) by post-translational processes (as hereinafter further described) or by the formation of disulphide bonds between cysteine residues; homologues thereof, such as disulphide-linked double-chained homologues, or sequences in which one or more amino acid is varied eg polymorphisms; isoforms; truncated forms or extended forms; and salts of any of the foregoing.

The polypeptides of the present invention have molecular weights in the range of from 7,000-17,000 Da, as measured by mass spectrometry, and preferably in the range 15,000-17,000 Da, which is greater than the contribution of all the amino acids in the sequences specifically described herein, owing to the presence of the above-mentioned post translational modifications

Especially preferred is a 130-amino acid polypeptide comprising both [SEQ ID NO: 50] and [SEQ ID NO: 60]. Particularly preferred is when the 130-amino acid polypeptide has a calculated molecular weight of approximately 14,500 Daltons and has a sequence selected from [SEQ ID NOS: 15-17]:

| | |
|---|---|
| V-E-F-Q-D-C-K-K-S-S-D-C-E-T-L-E-L-R-C-N-K-N-T-S-K-C-E-C-R-N-Q-V-C-P-R- | [SEQ ID NO: 15] |
| A-C-P-D-G-K-Y-K-L-D-E-Y-G-C-K-R-C-L-C-Q-G-C-N-E-A-Q-C-R-K-L-C-W-Y- | |
| G-F-T-T-D-E-N-G-C-E-S-Y-C-K-C-N-T-K-E-T-A-C-K-N-V-L-C-S-D-S-Y-Q-C-D- | |
| P-E-S-G-N-C-V-A-V-T-P-G-K-E-fI-D-Y-Y-S-Y-N-D-D-D-D-E-D-K; | |

| | |
|---|---|
| V-E-F-Q-D-C-K-K-S-S-D-C-E-I-L-E-L-R-C-N-K-N-I-S-K-C-E-C-R-N-Q-V-C-P-R- | [SEQ ID NO: 16] |
| A-C-P-D-G-K-Y-K-L-D-E-Y-G-C-K-R-C-L-C-Q-G-C-N-E-A-Q-C-R-K-L-C-W-Y | |
| G-F-T-T-D-E-N-G-C-E-S-Y-C-K-C-N-T-K-E-T-A-C-K-N-V-L-C-S-D-S-Y-Q-C-D | |
| P-E-S-G-N-C-V-A-V-I-P-G-K-E-H-D-Y-Y-S-Y-N-D-D-D-D-E-D-K; | |
| and | |
| V-E-F-Q-D-C-K-K-S-S-D-C-E-T-L-E-L-R-C-N-K-N-T-S-K-C-E-C-R-N-Q-V-C-P-R- | [SEQ ID NO: 17] |
| A-C-P-D-G-K-Y-K-L-D-E-Y-G-G-K-R-C-L-C-Q-G-C-N-E-A-Q-C-R-K-L-C-W-Y | |
| G-F-T-T-D-E-N-G-C-E-S-Y-C-K-C-N-T-K-E-T-A-C-K-N-V-L-C-S-E-S-Y-Q-C-D | |
| P-E-S-G-N-C-V-A-V-I-P-G-K-E-H-D-Y-Y-S-Y-N-D-D-D-E-D-K | |

Measurement of molecular weight by mass spectrometry, such as MALDI mass spectrometry, includes any post-translational modifications, so the aforesaid polypeptides can also include amino acid residues which have been modified by post-translational processes. The motif, N-T-S, occurs in positions 22-24 of, for example, SEQ ID NOS: 15-17, such as 16, and is a well-known site for potential N-linked glycosylation. Therefore, the invention includes polypeptides of the above sequences that are ‘derivable’ from the defined polypeptides by comprising a carbohydrate moiety linked to the 4-amide of asparagine-22 or to an equivalent position in the other sequences described. Such carbohydrate moieties may contain not more than 12 (ie ≦12) sugars or sugar derivatives in a single or branched chain. Furthermore, the encompassed polypeptides may be ‘derivable’ from the defined polypeptides by attachment of a sulphate or phosphato-moiety to the side chains of tyrosine residues. This is a common feature of proteins which originate in leeches where the aromatic ring of tyrosine can be modified, usually in the 4-position, by attachment of a sulphato- or phosphato-moiety. The invention therefore further includes sequences of the above formulae wherein one, two or all three tyrosine residues at positions 119, 120 and/or 122 of SEQID NOS: 15 to 17, such as 16, or the equivalent positions of the other sequences described, are sulphated or phosphated, especially sulphated.

By their resemblance to other similar polypeptides such as antistasin [Nutt E, Gasic T, Rodkey J et al. J Biol Chem 263: 10162-10167 (1988)], it can be deduced that polypeptides of the current invention are likely to have an “active site” between positions 63 and 64 of, for example, SEQ ID NOS: 15 to 17, such as 16. Since antistasin is cleaved in this position by its target protease; factor Xa, polypeptides of the current invention are also likely to be substrates for proteolysis at the putative “active site” [Dunwiddie C, Thornberry N A, Bull H G, et al. J Biol Chem 264: 16694-16699 (1989)]. In the case of antistasin, cleavage by the target protease, factor Xa, results in analogous polypeptides where a single peptide bond is cleaved. This cleavage produces a polypeptide comprising two chains held together by existing disulphide bonds, and the inhibitory effect on the target enzyme is retained. It is reasonable therefore to assume that two-chain homologues can exist, ‘derived’ from all the above sequences which comprise arginine at position 63 and lysine at position 64 or the equivalent arginine-lysine bond in the truncated sequences. The two chains are also held together by disulphide bonds and these homolgues are therefore likely to inhibit the alternative pathway of complement activation. In this case, the putative susceptible peptide bond links positions 63 and 64 of SEQ ID NO: 16, as shown by the arrow in the following fragment:

60 ↓ 70
........................N - E - A - Q - C - R - K - L - C - W - Y - G - F............

By ‘bioprecursor’ herein is meant a polypeptide which converts to a polypeptide of the present invention in vivo or otherwise under conditions of use. In particular, the invention encompasses the case where a so-called leader or signal sequence is present when the polypeptide is expressed in vivo, especially the case where the leader sequence comprises the natural leader sequence [SEQ ID NO: 38]:

M-K-Q-V-A-L-L-F-I-I-L-G-S-V-V-L-A [SEQ ID NO: 38];

or that of bee venom melittin:

M-K-F-L-V-N-V-A-L-V-F-M-V-V-Y-I-S-Y-I-Y-A [SEQ ID NO: 39].

Polypeptides described above with respect to [SEQ ID NOS: 15 to 17], respectively, comprising the natural leader sequence, are shown in [SEQ ID NOS: 10 to 12], respectively.

[SEQ ID NO: 10]

M K Q V A L L F I I L G S V V L A V E
F Q D C K K S S D C E T L E L R C N K
N T S K C E C R N Q V C P R A C P D G
K Y K L D E Y G C K R C L C Q C C N E
A Q C R K L C W Y G F T T D E N C C E
S Y C K C N T K E T A C K N V L C S D
S Y Q C D P E S G N C V A V T P G K E
H D Y Y S Y N D D D D E D K;

[SEQ ID NO: 11]

M K Q V A L L F I I L C S V V L A V E
E Q D C K K S S D G E T L E L R C N K
N T S K G E C R N Q V C P R A C P D G
K Y K L D E Y G C K R C L C Q G C N E
A Q C R K L C W Y G F T T D E N C C E
S Y C K G N T K E T A C K N V L C S D
S Y Q C D P E S G N C V A V I P G K E
H D Y Y S Y N D D D D E D K;
and

[SEQ ID NO: 12]

M K Q V A L L F I T L G S V V L A V E
F Q D C K K S S D C E T L E L R C N K
N T S K C E C R N Q V C P R A C P D G
K Y K L D E Y G C K R G L C Q C C N E
A Q C R K L C W Y C F T T D E N C C E
S Y C K C N T K E T A C K N V L C S E
S Y Q C D P E S G N C V A V I P G K E
H D Y Y S Y N D D D E D K.

Polypeptides as encompassed by this invention can advantageously form salts, preferably pharmaceutically acceptable salts, with any suitable non-toxic metal ion, or organic or inorganic acid or base. Examples of such inorganic acids include hydrochloric, hydrobromic, sulphuric and phosphoric acid, and acid metal salts such as sodium monohydrogen orthophosphate and potassium hydrosulphate. Examples of organic acids include mono-, di- and tri-carboxylic acids such as acetic, glycolic, lactic, pyruvic and sulphonic acids, or the like. Examples of bases include ammonia; primary, secondary and tertiary amines; and quaternary ammonium salts. Polypeptides of this invention can be prepared from leech species of the order Rhynchobdellida and more particularly those of the genus Placobdella, especially of the species Placobdella pupillifera. Alternatively, the polypeptides can be synthesised chemically or produced by transgenic organisms carrying DNA sequences which encode them.

Polypeptides of this invention may be extracted from leech tissue or secretions by, for example, homogentisation of substantially the whole leech, or the salivary glands or the proboscis or the like, in a suitable buffer. The present invention therefore further provides an inhibitor of the alternative complement pathway derivable from leech tissue or secretions. The term ‘derivable’ as used in this context encompasses material that is directly derived, such as by extraction or purification, as well as material that is indirectly derived by being a physically-, chemically- or genetically-engineered product of a process applied to material directly derived or converted to a chemically-modified derivative. The polypeptides are typically extracted or purified using a combination of known techniques such as, for example, ion exchange, gel filtration and/or reversed phase chromatography.

Leeches of the same genus, or even the same species, often have more than one polypeptide in their salivary glands that have similar biochemical effects and highly homologous amino acid sequences. In addition, in the same species of leech, several different isoforms of a particular polypeptide may exist, which differ by only a few amino acids. This invention therefore includes polypeptide derivatives which inhibit the alternative complement pathway and in which no more than 20% of the amino acids in the polypeptide chain differ from those listed. The figure of 20% is based on the fact that many homologues of another leech protein, hirudin, occur naturally in Hirudo medicinalis and are described in the literature; the most diverse of these differ in 15 of the 65 amino acids in the polypeptide chain. Furthermore, one or more additional amino acids may be interposed in the polypeptide chain (extended forms), provided that they do not interfere with the pharmacological activity of the polypeptide. This invention also encompasses truncated forms of the polypeptide, where one or two of the N- or C-terminal amino acids are deleted.

A polypeptide encompassed by this invention can also be prepared by providing a host, transformed with an expression vector comprising a DNA sequence encoding the polypeptide under such conditions that the polypeptide is expressed therein, and optionally isolating the polypeptide thus obtained. This approach is typically based on obtaining a nucleotide sequence encoding the polypeptide it is wished to express, and expressing the polypeptide in a recombinant organism. The cultivation of the genetically-modified organism leads to the production of the desired product displaying full biological activity. The present invention therefore also comprises a polypeptide produced by a recombinant DNA technique, which polypeptide is one encompassed above. The invention further comprises a synthetic, or protein-engineered, polypeptide encompassed above.

Accordingly, the present invention further provides a nucleic acid sequence, in particular an isolated, purified or recombinant nucleic acid sequence; comprising:

(a) a nucleic acid sequence encoding a polypeptide encompassed by the present invention;

(b) a sequence substantially homologous to or that hybridises to sequence (a) under stringent conditions;

(c) a sequence substantially homologous to or that hybridises to the sequences (a) or (b) but for degeneracy of the genetic code; and

(d) an oligonucleotide specific for any of the sequences (a), (b) or (c) above.

By “substantially homologous” herein is meant that the nucleic acid sequence has at least 80% identity of its nucleotide bases with those of sequence (a), in matching positions in the sequence, provided that up to six bases may be omitted or added therein. Preferably, the sequence has at least 90% homology and more preferred are sequences having at least 95% homology with the sequence (a). Such homologous sequences encode a protein having substantially the same biological activity as the proteins of the invention.

Oligonucleotides “specific for” any of these nucleic acid sequences (a) to (c) above are useful for identifying and isolating the biologically active peptides of this invention, and comprise a unique sequence encoding a unique fragment of the amino acid sequence of the peptide. These include [SEQ ID NOS: 33 to 36], defined hereinbelow in Example 4; and [SEQ ID NO: 37], defined hereinbelow in Example 5.

In particular, the present invention provides a nucleic acid sequence as defined above, wherein the sequence is a DNA or RNA sequence, such as cDNA or mRNA. More particularly, the present invention provides a DNA sequence identified herein by [SEQ ID NO: 6], which sequence corresponds with the polypeptide identified herein as [SEQ ID NO: 10] which it will be appreciated is the same as the polypeptide listed as [SEQ ID NO: 15] including its signal sequence [SEQ ID NO: 38]; a DNA sequence listed herein by [SEQ ID NO: 7], which sequence corresponds with the polypeptide identified herein as [SEQ ID NO: 11] comprising the polypeptide [SEQ ID NO: 16] plus the signal sequence and; DNA sequences [SEQ ID NO: 8] and [SEQ ID NO: 9] which are polymorphic with respect to each other and both encode the polypeptide identified herein as [SEQ ID NO: 12] comprising the polypeptide [SEQ ID NO: 17] plus the signal sequence. Especially preferred is the DNA sequence SEQ ID NO: 13 which encodes the polypeptide [SEQ ID NO: 16] including the bee venom melittin signal sequence [SEQ ID NO: 39

Therefore, the present invention further provides a method for the preparation of a polypeptide according to the present invention, which method comprises:

(a) isolation and/or purification from whole leeches, or tissue or extracts from a Rhynchobdellida leech, preferably a Placobdella leech, such as P. papillifera; or

(b) expression of a nucleic acid sequence encoding the polypeptide and, optionally, isolation and/or purification of the resulting polypeptide.

The present invention further provides: a recombinant construct comprising any nucleic acid sequence according to the invention; a vector comprising such a construct; and a host transformed or transfected by such a vector. The present invention therefore further provides a cell, plasmid, virus, live organism or other vehicle that has been genetically or protein-engineered to produce a polypeptide according to the present invention, said cell, plasmid, virus, live organism or other vehicle having incorporated expressably therein a sequence as disclosed herein. Such cells may include animal, such as mammal, for example human or humanised cells, for use in gene therapy to treat or prevent conditions such as those mentioned herein.

In another aspect, the present invention provides a method for the treatment or prevention of a condition or disorder mentioned herein, wherein the polypeptide is administered by means of being expressed in the cells of the patient, which cells have incorporated expressably therein a nucleic acid sequence coding for the polypeptide. Alternative to gene therapy, the polypeptides of the invention may be administered as a pharmaceutical formulation.

Accordingly, the present invention provides the use of a polypeptide described herein or a nucleic acid sequence coding for the polypeptide in medicine, including gene therapy; and also the use of such a polypeptide in the manufacture of a medicament.

Therefore, according to a further aspect of the present invention, there is provided a pharmaceutical formulation comprising a polypeptide according to the invention (as described above) and a pharmaceutically acceptable carrier therefor. The term “pharmaceutically acceptable carrier” as used herein should be taken to mean any inert, nontoxic, solid or liquid filler, diluent or encapsulating material, or other excipient, which does not react adversely with the active ingredient(s) or with a patient.

Such formulations and carriers are well known in the art and include pharmaceutical formulations that may be, for example, administered to a patient systemically, such as parenterally, or orally or topically.

The term ‘parenteral’ as used here includes subcutaneous, intravenous, intramuscular, intra-arterial and intra-tracheal injection, and infusion techniques. Parenteral formulations are preferably administered intravenously, either in bolus form or as a continuous infusion, or subcutaneously, according to known procedures. Preferred liquid carriers, which are well known for parenteral use, include sterile water, saline, aqueous dextrose, sugar solutions, ethanol, glycols and oils.

Tablets and capsules for oral administration may contain conventional excipients such as binding agents, fillers, lubricants, wetting agents, and the like. Oral liquid preparations may be in the form of aqueous or oily suspensions, solutions, emulsions, syrups, elixirs or the like, or may be presented as a dry product for reconstitution with water or other suitable vehicle for use. Such liquid preparations may contain conventional additives, such as suspending agents, emulsifying agents, non-aqueous vehicles and preservatives.

Formulations suitable for topical application may be in the form of aqueous or oily suspensions, solutions, emulsions, gels or, preferably, emulsion-based ointments.

Unit doses of the pharmaceutical formulations according to the invention may contain daily required amounts of the polypeptides, or sub-multiples thereof to make a desired dose. The optimum therapeutically-acceptable dosage and dose rate for a given patient (which may be a mammal, such as a human) depend on a variety of factors, such as the potency of the active ingredient(s); the age, body weight, general health, sex and diet of the patient; the time and route of administration; rate of clearance; the object of the treatment (for example, treatment or prophylaxis); and the nature of the disease to be treated.

It is expected that systemic doses in the range of from 0.005 to 50 mg/kg body weight, preferably of from 0.005 to 10 mg/kg and more preferably 0.01 to 1 mg/kg, will be effective. According to the nature of the disease being treated, one single dose may comprise in the range of from 0.005 to 10 mg/kg body weight active ingredient, whether applied systemically or topically.

The polypeptides have the ability to inhibit alternative complement-mediated haemolysis of non-antibody-coated erythrocytes and can also inhibit the complement-mediated haemolysis of guinea pig erythrocytes when induced by cobra venom factor. The polypeptides are highly selective for the alternative pathway because they have no effect on antibody-mediated activation of complement by the classical pathway at concentrations significantly more than 100 times those which inhibit the alternative pathway by 50%. Moreover, they have no significant effect on the clotting time of human plasma, measured by either the prothrombin time or the activated partial thromboplastin time, nor on the time required for streptokinase to lyse human plasma clots at more than 100 times the concentration which inhibits the alternative pathway by 50%. Further details of these experiments are given below in the Examples.

It therefore follows that the polypeptides of this invention selectively inhibit one or more steps in the alternative pathway of complement activation. Since they also inhibit C3a production when human serum is activated by cobra venom factor, the most likely site of action is by inhibition of either or both of the proteases, factor D or the C3bBb complex, which are essential mediators of the alternative pathway. There is therefore provided, as a farther aspect of this invention, a polypeptide which interacts with (eg inhibits or binds to) complement factor D and/or the C3bBb complex.

The polypeptides of this invention can potentially be used to inhibit detrimental activation of the alternative complement pathway in patients, for example: in haemodialysis or cardiopulmonary bypass; when in-dwelling catheters or intra-arterial stents are present; in rejection of transplanted organs or tissues; in various auto-immune diseases such as lupus arthritis; rheumatoid arthritis; or glomerulonephritis; nephritis; nephropathy; sepsis; or injury caused to tissues by reperfusion after an ischaemic period, such as happens in heart attacks and strokes. Other conditions such as anaphylaxis, asthma, skin reactions, infections, sickle cell anaemia and haemolytic anaemia which are associated with activation of complement, as adjudged by the appearance of activated components or their complexes in biological fluids and/or their deposition in diseased tissues, may also potentially be treated with polypeptides of this invention.

A further aspect of this invention therefore provides a covalent complex of a polypeptide encompassed by this invention with the surface of a prosthesis or extracorporeal circulation which is exposed to blood, in order to prevent the initiation of complement activation.

Furthermore, said polypeptides may be used in combination with immunosuppressant agents which may advantageously decrease transplant rejection in synergistic fashion or with steroidal or in combination with non-steroidal anti-inflammatory drugs to increase their efficacy. The term “in combination” should be taken to mean the simultaneous or sequential administration of a polypeptide according to the invention, together with one or more immunosuppressant and/or anti-inflammatory drug(s).

The present invention therefore further provides:

(a) the use of a polypeptide of this invention in therapy;

(b) the use of a polypeptide of this invention in the preparation of a medicament;

(c) a method for the treatment or prevention of a condition in a patient, which condition involves activation of the alternative complement pathway, which method comprises administration to said patient of a non-toxic, inhibitory amount of a polypeptide of the invention; and

(d) the use of a polypeptide of this invention in the selective inhibition of the alternative pathway of complement activation, compared to inhibition of the classical and/or coagulation (blood clotting) pathways.

The invention will now be further described with reference to the following Examples.

### EXAMPLE 1

### Alternative Complement Inhibitory Activity of Placobdella papillifera Extracts

In order to identify complement inhibitory factors, leech extracts were prepared and assayed in a haemolytic assay for alternative complement (AP₅₀). For preparation of an extract, the salivary glands of one specimen of Placobdella papillifera were homogenised in phosphate buffered saline (PBS) (pH 7.4, 0.5 ml) (from Sigma-Aldrich Company Ltd., Fancy Road, Poole, Dorset, UK) and centrifuged at 13,000 rpm for 3 min to remove tissue debris. Rabbit erythrocytes in acid citrate dextrose (ACD) (20% v/v) (from Harlan Sera-Lab Ltd., Dodgeford Lane, Belton, Loughborough, Leicestershire, UK) were washed twice in ice-cold ACD (Sigma-Aldrich), and three times in Gelatin Veronal Buffer (GVB) (from Sigma-Aldrich, UK) supplemented with 7 mM MgCl₂, 10 mM EGTA (Sigma-Aldrich trade mark) pH 7.2 (hereinafter referred to as VCM-MEG) by suspension and centrifugation at 2,000 rpm for 10 min. Erythrocytes were re-suspended (1% v/v) in VCM-MEG prior to use. Freeze-dried human plasma (5 ml) (from Sigma-Aldrich, UK) was reconstituted in 5 ml of ice-cold VCM-MEG and diluted (1 in 4) for the assay.

The haemolytic assay was carried out on microtitre plates which contained PBS or extract supernatant (0.03 ml), diluted plasma (0.075 ml), rabbit erythrocytes (0.075 ml) and VCM-MEG (0.045 ml). The plate was incubated for 45 min at 37° C., then EDTA (0.2 M, 0.0225 ml) was added to the haemolytic wells to stop the reaction. The samples were centrifuged and the absorbance of the supernatant read in a microtitre plate reader at 405 nm. The degree of haemolysis in the test wells was assessed by comparison with fully-lysed cells, which had been prepared in water, and non-lysed cells, where plasma had been substituted with VCM-MEG. The concentration of plasma (complement) used typically caused 70% lysis of the erythrocytes, compared to the buffer control. The leech extract reduced this per centage, indicating that it possessed alternative complement inhibitory activity (Table 1).

TABLE 1

| | Absorbance | |
|---|---|---|
| Extract | at 405 nm | % Lysis |
| Fully lysed cells | 1.053 | 100.0% |
| PBS (control) | 0.739 | 70.2% |
| Placobdella papillifera | 0.021 | 2.0% |

### EXAMPLE 2

### Extraction/Purification of Active Polypeptide from Placobdella papillifera

In order to purify one example of active polypeptide, a combination of ion exchange chromatography and gel filtration was used. Salivary glands from 50 specimens of Placobdella papillifera were homogenised in 20 mM Tris HCl buffer (pH 8.0, 6 ml). After centrifugation, the supernatant was applied to a HiPrep®16/10 Q XL column (from Amersham Pharmacia Biotech UK Ltd., Amersham Place, Little Chalfont, Buckinghamshire, UK), which had been equilibrated in 20 mM Tris HCl (pH 8.0). Bound proteins were eluted with a linear gradient from the starting buffer to 20 mM Tris HCl (pH 8.0) containing 1 M NaCl over 10 column volumes and detected by absorbance at 280 nm. Fractions were assayed by the AP₅₀ method as described in Example 1. Fractions exhibiting inhibitory activity eluted between approximately 0.56 and 0.83 M NaCl.

These fractions were dialysed against 20 mM sodium formate buffer (pH 4.0) and applied to a 1 ml column of HiTrap® SP Sepharose Fast Flow (from Amersham Pharmacia Biotech, UK) and eluted with a linear gradient from 20 mM sodium formate (pH 4.0) to the same buffer containing 1 M NaCl over 10 column volumes, and protein was detected by absorbance at 280 nm. The inhibitory activity, as measured by the AP₅₀ assay, eluted between 0.1 and 0.63 M NaCl.

Fractions containing the active peak were lyophilised, reconstituted in water (2 ml) and applied to an XK 16/30 column of Superdex™ 75 prep grade (from Amersham Pharmacia Biotech, UK) equilibrated in PBS (pH 7.4). The inhibitory activity, as measured by the AP₅₀ assay, eluted at approximately 0.49 column volumes.

The fractions containing the inhibitory activity were dialysed against 20 mM sodium formate (pH 4.0) and applied to a Mini S® PE 4.6/5 column (from Amersham Pharmacia Biotech, UK). A linear gradient from 20 mM sodium formate (pH 4.0) to the same buffer containing 1 M NaCl over 25 column volumes eluted one major peak which contained the inhibitory activity, at approximately 0.22 M NaCl.

Application of the fractions containing the major peak on to an XK 16/30 column of Superdex™ 75 prep grade, eluted with PBS (pH 7.4), resulted in a discrete peak at approximately 0.49 column volumes, which contained the inhibitory activity. The peak contained a single component on reversed phase HPLC on a 1 ml RESOURCE™ RPC column (from Amersham Pharmacia Biotech, UK).

### EXAMPLE 3

### Amino Acid Sequence and molecular weight of Active Polypeptide

The active fraction, purified according to Example 2, was subjected to amino acid sequencing. There was a clean, clear amino acid sequence from the N-terminus. The sequence [SEQ ID NO: 1] of the first 21 amino acids was found to be:

V-E-F-Q-D-X-K-K-S-S-D-X-E-T-L-E-L-R-X-N-K- [SEQ ID NO: 1]

wherein each X represents a single amino acid that could not be identified and therefore each X could be the same or different.

In order to identify more of the amino acid sequence, lyophilised samples of the active fraction from Example 2 were digested with proteases. Thus, approximately 0.01 mg of the lyophilised fraction was dissolved in 8 M urea, 0.4 M ammonium bicarbonate (25 μl), 0.045 M dithiothreitol (5 μl) was added and the sample incubated at 50° C. for 15 min. Cysteine residues were carboxymethylated by addition of 0.1 M iodoacetamide (5 μl). The samples were diluted with water (60 μl) and either 0.1 μg/ml Lys-C or Asp-N endoprotease (4 μl) (from Sigma-Aldrich, UK) was added and the samples incubated at 37° C. for 24 h. The reaction was stopped by freezing at −20° C. Cleaved peptides were separated by reversed phase HPLC on a 1 ml RESOURCE™ RPC column (from Amersham Pharmacia Biotech, UK) and the amino acids sequenced. The following peptides were identified:

from Lys-C endoprotease digestion [SEQ ID NO: 2]:

L-X-W-Y-G-F-T-T-

from Asp-N endoprotease digestion [SEQ ID NO: 3]:

X-E-Y-G-X-K-R-X-L-X-Q-G-X-N-E-A-Q-X-R-K-

wherein each X represents a single amino acid that could not be identified and therefore each X could be the same or different.

The molecular weight of the active fraction was estimated to be approximately 16,180 Daltons by MALDI mass spectrometry.

### EXAMPLE 4

### Cloning and Sequencing of cDNA Encoding Active Polypeptide

The available amino acid sequences identified in Example 3 (ie [SEQ ID NO: 3]) allowed oligonucleotide primers to be designed so that the cDNA encoding these polypeptides could be cloned from frozen salivary glands and sequenced. Frozen salivary glands (approx. 150 mg) from Placobdella papillifera were thawed in guanidinium thiocyanate lysis solution (0.5 ml) as described in the Micro (A) Pure kit (from Ambion Inc., 2130 Woodward Street, Austin, Tex., USA). Following homogenisation, dilution buffer (1 ml) (from Ambion, USA) was added, mixed and the supernatant collected following centrifugation at 12,000 g for 15 min at 4° C. Poly A+ RNA was extracted on and eluted from oligo dT resin by the method described in the Ambion Micro (A) Pure kit. A complementary DNA strand was constructed by reverse phase transcription using a cDNA amplification kit (from Clontech Laboratories UK Ltd., Unit 2, Intec 2, Wade Road, Basingstoke, Hampshire, UK) and the second strand cDNA was synthesised by T4 DNA polymerase (also from Clontech, UK). The resulting double-stranded DNA was extracted in phenol/chloroform, precipitated in ethanol and re-dissolved in sterile water.

Isolation of the 5′ and 3′ cDNA ends was facilitated by ligation of Marathon Adaptor sequence (from Clontech). The 5′ end was identified using polymerase chain reaction (PCR) with Taq polymerase (from Life Technologies Ltd., 3 Fountain Drive, Inchinnan Business Park, Paisley, UK), AP1 primer (from Clontech, UK) and degenerate primers:

GC(CT) TC(AG) TT(AG) CA(AGCT) CC(CT) TG(AG) CA [SEQ ID NO: 33]

designed from the Asp-N endopeptidase peptide described in Example 3. A DNA fragment of approximately 300 base pairs was isolated and purified using the Qiagen QIAquick PCR Purification Kit and, in order to increase the amount available for sequencing, it was ligated to plasmid vector pCR®2.1—TOPO™ (TOPO™ TA Cloning® kit) (from Invitrogen BV, PO Box 2312, 9704 CH Groningen, The Netherlands). The resultant recombinant plasmids were introduced into competent Escherichia coli (TOP10) and stocks of recombinant clones and plasmid DNA generated using the QIAprep Spin Miniprep Kit. Plasmids were sequenced and the sequence containing that predicted from the peptides [SEQ ID NOS: 1 and 3] was identified as [SEQ ID NO: 4].

This permitted the design of gene-specific primers, which were used to obtain the 3′ ends of the peptides. An asymmetric PCR approach was used to enrich the specific product using only one primer by incubating the cDNA from above (5 μl), 10×PCR reaction buffer (from Life Technologies, UK) (5 μl), 10 mM dNTP mix (1 μl), 1.5 mM MgCl₂ (1.5 μl), 0.01 mM gene-specific primer (1 μl):

GGG GTC GGT AGT TTT GGC GGT AGA G [SEQ ID NO: 34]

Taq polymerase (0.5 μl) and water (36 μl). The reaction mixture was denatured at 94° C. for 2 min and cycled in a Techne Genius DNA Thermal Cycler as follows: 94° C. for 30 sec, 65° C. for 30 sec and 72° C. for 1 min for 15 cycles and finally at 72° C. for 10 min. The reaction mixture was then used as the template in a PCR reaction using both Adaptor Primer 1 (from Clontech, USA) and the gene-specific primer under similar conditions. A DNA fragment of approximately 600 base pairs was isolated and purified, ligated into plasmid vector pCR®2.1-TOPO™ (TOPO™ TA Cloning® kit) (from Invitrogen, The Netherlands) and stocks of recombinant clones were generated as above. Plasmids were sequenced, and sequences containing those predicted from the amino acid sequences [SEQ ID NOS: 1 and 3] identified [SEQ ID NO: 5]. From the two cDNA sequences identified above ([SEQ ID NO: 4] and [SEQ ID NO: 5]), it was possible to deduce the amino acid sequence of a polypeptide corresponding to one embodiment of the invention (namely, [SEQ ID NO: 10]).

Using the DNA sequences identified, two more gene-specific primers were synthesised in order to amplify the entire coding region of the peptides from the adaptor-ligated cDNA These were used in the following PCR reaction: cDNA from above (5 μl), 10×PCR reaction buffer (5 μl from Life Technologies), 10 mM dNTP mix (1 μl), 1.5 mM MgSO₄ (1 μl), 0.01 mM gene specific primer (1 μl):

CGG GCA GGT ATC ATA ATG [SEQ ID NO: 35]

0.011 mM gene specific primer (1 μl):

AGT CGT TCG TTC GTT TTC [SEQ ID NO:36]

Platinum Pfx DNA polymerase (0.5 μl) (from Life Technologies) and water (35.5 μl). The reaction mixture was denatured at 94° C. for 2 min and cycled in a Techne Genius DNA Thermal Cycler as follows: 94° C. for 30 sec, 45° C. for 30 sec and 68° C. for 1 min for 30 cycles and finally at 68° C. for 10 min. Three independent PCR reactions were performed, and specific DNA fragments of approximately 500 base pairs were isolated and purified as above. Each of these fragments was ligated to plasmid vector pCR® Blunt (using the ZeroBlunt®) PCR Cloning Kit from Invitrogen, NL) and the resultant recombinant plasmids were introduced into competent E. coli (TOP10). Stocks of recombinant clones and plasmid DNA were generated as before using the QIAprep Spin Miniprep Kit. The three plasmids resulting from the three independent PCR reactions were sequenced and three variant DNA sequences, each encoding amino acid sequences similar to [SEQ ID NO: 10] were identified (namely, [SEQ ID NO: 7]), which encodes polypeptide SEQ ID NO: 11 and [SEQ ID NOS: 8 and 9], which both encode polypeptide [SEQ ID NO: 12]).

By comparing the deduced amino acid sequences ([SEQ ID NOS: 10-12]) with those obtained from the isolated natural polypeptide in Example 3, a signal sequence comprising:

M-K-Q-V-A-L-L-F-I-I-L-G-S-V-V-L-A- [SEQ ID NO:38]

was identified, which is presumably removed before the protein is secreted from the cells that express the protein.

### EXAMPLE 5

### Preparation of Recombinant Active Polypeptide

In order to express one of the polypeptides in baculovirus-infected insect cells, the signal sequence was replaced with that of honey bee melittin. Oligonucleotides were designed wherein the natural leader or signal sequence was replaced by the melittin signal sequence in the SEQ ID NO 7. These oligonucleotidess were:

(melittin specific): AGA ATT CTA AAT ATG AAA TTC TTA GTC AAC GTT GCC CTT GTT TTT ATG GTC GTA TAC ATT TCT TAC ATC TAT GCG GTA GAG TTT CAA GAT TGC AAG; [SEQ ID NO: 40]

(as disclosed in Tessier DC. Thomas DY. Khouri HE. Laliberte F. Vemet T. Gene 98:177-83 (1991) but having appended thereto (indicated in bold typeface) a polypeptide gene-specific sequence); and

(polypeptide gene-specific):

ACT GCA GAG TCG TTC GTT CGT TTT CAT TTA TC) [SEQ ID NO: 37]

Using the BAC-TO-BAC Baculovirus Expression Systems Instruction Manual, Gibco BRL (from Life Technologies), this construct was cloned into pFastBac1 and the sequence confirmed ([SEQ ID NO: 13)). E. coli DH10 cells were transformed with the recombinant pFastBac1. Transposition was confirmed by blue/white selection. Single colonies were selected and Bacmid DNA isolated. Sf21 cells (available from the Nerc, Oxford UK) in 47.5% Ex-cell 401 (from AMS Biotechnology UK Ltd., 185 A & B Milton Park, Abingdon, Oxfordshire, UK) and 47.5% TC100 (from Life Technologies) containing 5% heat-inactivated foetal bovine serum (from Life Technologies) in shaker flasks were transfected with purified recombinant Bacmid DNA in the presence of Lipofectin (from Life Technologies) using standard methods [as described in, for example, King L A & Possee R D (1992) The Baculovirus Expression System, published by Chapman & Hall], and the recombinant virus was collected 7 days post-transfection.

Transfection mixes were then used to infect Sf21 cells in serun-free medium (Ex-cell 401) harvested 96 h post-infection, and the culture media assayed in an AP₅₀ assay as described in Example 1. The viral stock which gave the most inhibitory effect was selected, and high titre stocks were generated in Sf21 cells (100 ml) cultured at 1.92 cells/ml in Ex-cell 401, TC 100, 5% foetal bovine serum by infection at 0.5 multiplicity of infection. Antibiotics were added at 50 IU/ml penicillin, 5 μg/ml streptomycin. Virus was harvested at 7 days and titred by plaque assay [King L A & Possee R D (1992), ibid]. The viral stock was found to be 2×10⁸ pfu/ml. Infection of Sf21 cells in Ex-cell 401 with the viral stock at multiplicity of infection 10 resulted, at 96 h post-infection, in expression of 6.8 mg/litre of the polypeptide measured by inhibition of the AP₅₀ assay, in comparison with a preparation of the natural polypeptide of known concentration. Infection of HiS cells (available from Invitrogen, NL) in Ex-cell 405 in the same way resulted in expression of 41.8 mg/litre of the inhibitory polypeptide. The DNA sequence [SEQ ID NO: 13] encodes a mature (ie after cleavage of the mellitin signal sequence) polypeptide [SEQ ID NO: 16].

### EXAMPLE 6

### Purification of Recombinant Active Polypeptide

In order to purify the recombinant polypeptide from Example 5, a combination of ion exchange and gel filtration column chromatography was carried out. Culture medium from both Sf21 cells and from HiS cells (total volume 410 ml) was pooled and dialysed against 50 mM Tris HCl, 0.3 M NaCl (pH 8.0), adjusted to pH 8.0 with NaOH and centrifuged at 2,500 rpm for 3 min to remove any insoluble material. This was applied to a HiPrep®16/10 Q XL column (from Amersham Pharmacia Biotech, UK) which had been equilibrated in 20 mM Tris HCl (pH 8.0). Bound proteins were eluted with a step gradient from 20 mM Tris HCl (pH 8.0) to the same buffer containing 0.3 M NaCl. Protein elution was detected by absorbance at 280 nm. When the absorbance returned to baseline, a linear gradient from 50 mM Tris HCl, 0.3 M NaCl (pH 8.0) to 20 mM Tris HCl pH 8.0 containing 1 M NaCl over 10 column volumes was started. Fractions were assayed in the AP₅₀ method described in Example 1. The activity eluted between 0.46 and 0.72 M NaCl.

The fractions containing the inhibitory activity were dialysed against 20 mM sodium formate buffer (pH 4.0) and applied to a 1 ml column of HiTrap® SP Sepharose Fast Flow (from Amersham Pharmacia Biotech, UK) and eluted with a linear gradient from 20 mM sodium formate (pH 4.0) to the same buffer containing 1 M NaCl over 15 column volumes, and protein was detected by absorbance at 280 nm. The inhibitory activity, as measured by the AP₅₀ assay, eluted between 0.30 and 0.50 M NaCl.

The active fractions were applied to an XK 16/30 column of Superdex™ 75 prep grade (from Amersham Pharmacia Biotech, UK) equilibrated in PBS (pH 7.4). The inhibitory activity, as measured by the AP₅₀ assay, eluted at approximately 0.45 column volumes.

N-terminal amino acid sequencing of the pure recombinant polypeptide confirmed the amino acid sequence V-E-F-Q-D- consistent with that expected of [SEQ ID NO 16].

### EXAMPLE 7

### Alternative Complement Pathway Inhibitory Potency of Active Polypeptides

The potency of the described polypeptides as inhibitors of the alternative pathway of complement activation was determined in the AP₅₀ assay as described in Example 1. The inhibitory fractions inhibited the lysis of erythrocytes, indicating an inhibitory effect on the alternative pathway of complement activation. By varying the concentration of the polypeptide, the IC₅₀ was determined (Table 2).

TABLE 2

| Test Sample | IC₅₀ (ng/ml) |
|---|---|
| Natural polypeptide from Example 2 | 39.6 |
| Recombinant polypeptide from Example 6 | 35.9 |

### EXAMPLE 8

### Selectivity of Active Polypeptides for Alternative Complement Pathway

Polypeptides of this invention are specific for the alternative pathway of complement activation, since there is no effect on the classical pathway as adjudged by a lack of effect on the haemolytic (CH₅₀) assay at concentrations of up to 500 times the IC₅₀ in the AP₅₀ assay.

Preparation of erythrocytes for the CH₅₀ assay was carried out as follows. Sheep erythrocytes (from Oxoid Ltd., Wade Road, Basingstoke, Hampshire, UK) were washed 3 times in Barbitone Complement Fixation Test diluent (CFT) (from Oxoid, UK) supplemented with 0.1% (w/v) gelatin (CFT-G) by centrifugation at 2,000 rpm for 10 min and re-suspension. The erythrocytes were coated with antibody by the following procedure: sheep haemolysin (from Harlan Sera-Lab), diluted 1:200 in CFT-G (4 ml), was added to erythrocytes diluted 1:4 in CFT-G (4 ml) and incubating at 37° C. for 30 min then at 0° C. for a further 30 min with periodic mixing. The coated erythrocytes were washed twice in CFT-G, re-suspended in CFT-G supplemented with 2.5% (w/v) glucose and 0.1% (w/v) sodium azide, and diluted in CFT-G to give an absorbance at 405 nm of 0.7 when fully lysed.

For the assay, blank microtitre plate wells (0% haemolysis) contained CFT-G (0.2 ml) plus coated erythrocytes (0.05 ml). Wells containing water (0.2 ml) and coated erythrocytes (0.05 ml) gave an absorbance value for 100% haemolysis. Test wells contained: CFT-G (0.1 ml); dilutions of the active fraction from Example 2 or PBS (0.05 ml); human serum (complement) diluted in CFT-G to give approximately 75% haemolysis (0.05 ml) and coated erythrocytes (0.05 ml). The plate was covered and incubated at 37° C. for 1 h and centrifuged at 1,000 rpm for 3 min. Supernatants (0.2 ml) were transferred to a microtitre plate and absorbance was read at 405 nm.

The absorbances of wells containing the active fraction from Example 2 at concentrations ranging from 0.04 pg/ml to 340 ng/ml did not differ significantly from those containing PBS (0.227±0.012 versus test sample: 0.233±0.014 respectively, P>0.05. T test, not significant). Moreover, there was no correlation of absorbance with the concentration of the active fraction demonstrating that there was no effect on the CH₅₀ assay under these conditions. Similarly, there was no effect of the recombinant polypeptide from Example 6 in the range 36.9 pg/ml to 19.6 μg/ml (PBS: 1.088±0.044 versus test sample: 1.065±0.009, P>0.05. T test, not significant).

### EXAMPLE 9

### Inhibition of CVF-Induced Complement-Mediated Erythrocyte Haemolysis

In addition to the classical and alternative pathways, the complement cascade can be activated by cobra venom factor (CVF). CVF forms a complex with factor B resulting in an active C3 and CS convertase which by-passes both the classical and alternative pathway activation steps [Vogel C-W, Muller-Eberhard H J. J Immunol Methods 73: 203-220 (1984)]. A distinguishing feature of polypeptides of this invention is that they potently inhibit complement-mediated erythrocyte haemolysis caused by CVF.

Guinea pig erythrocytes (from Harlan Sera-Lab) were washed three times in GVB (from Sigma-Aldrich) and diluted (1:5) before use. Assay samples contained: guinea pig serum (from Harlan Sera-Lab) (0.02 ml); washed guinea pig erythrocytes (from Harlan Sera-Lab) (0.02 ml); and serial dilution of test sample dissolved in PBS (0.02 ml). Haemolysis was stimulated by addition of CVF (from Quidel, Appligene-Oncor-Lifescreen, Unit 15, The Metro Centre, Dwight Road, Watford, Hertfordshire, UK) (0.02 ml of 130 °g/ml). Following incubation for 30 min at 37° C., the reaction was stopped by adding ice-cold GVB (0.5 ml). After centrifugation, supernatants (0.1 ml) were transferred to a microtitre plate and absorbance read at 405 nm.

The polypeptides inhibited complement mediated haemolysis induced by CVF with similar IC₅₀ (Table 3).

TABLE 3

| Test Sample | IC₅₀ (ng/ml) |
|---|---|
| Natural polypeptide from Example 2 | 970 |
| Recombinant polypeptide from Example 6 | 1217 |

### EXAMPLE 10

### Inhibition of CVF-Induced Factor D by Active Polypeptides

In addition to its ability to stimulate haemolysis, cobra venom factor leads to activation of the alternative pathway leading to the generation of C3a, a peptide fragment that is cleaved from C3. C3a production can be measured with an enzyme immunoassay (from Quidel, UK).

Assay samples contained: serial dilutions of test sample dissolved in PBS (0.02 ml); GVB (from Sigma-Aldrich) (0.02 ml), and human serum (0.02 ml). Complement was activated by addition of CVF (from Quidel) (0.02 ml), and samples incubated for 30 min at 37° C. A non-activated serum control, where PBS was substituted for CVF, was incubated for 30 min at 4° C. All samples were diluted 1 in 10,000 in sample buffer (from Quidel) prior to measuring C3a by the ELISA kit (from Quidel). Both natural and recombinant polypeptides inhibited complement generated C3a induced via CVF (Table 4).

TABLE 4

| Test Sample | IC₅₀ (ng/ml) |
|---|---|
| Natural polypeptide from Example 2 | 95 |
| Recombinant polypeptide from Example 6 | 138 |

The biochemical pathway leading to C3a production in serum stimulated with cobra venom factor is believed to involve cleavage of factor B in the complex C3bB by the protease, factor D, and cleavage of C3 by the proteolytic action of the resulting C3bBb complex. Since a large molar concentration of CVF was used, it is a reasonable assumption that a large part of the factor B in the above example was converted to the active factor Bb. From literature values for serum, the factor B concentration in the assay was estimated to be approximately 540 nmoles/litre whereas that of factor D was approximately 20 mmoles/litre. Since the stoichiometry for inhibition cannot be less than one mole of polypeptide inhibitor to inhibit one mole of enzyme, the concentration of inhibitor that inhibits by 50% cannot be less than 50% of the enzyme concentration. In this example, the concentration of the polypeptide to inhibit the assay by 50% was 5.9 nmoles/litre for the natural polypeptide and 8.6 nmoles/litre for the recombinant polypeptide. Since these IC₅₀s are approximately equivalent to 50% of the concentration of the factor D but less than 2% of that of the factor B or other components in the assay, it is most likely that the polypeptides act in this assay primarily by inhibiting factor D.

### EXAMPLE 11

### Direct Interaction of Active Polypeptides with Factor D

The direct binding of the described polypeptides to factor D was investigated by surface plasmon resonance analysis on a Biacore® X analytical system (from Biacore AB, Uppsala, Sweden).

Human factor D (Calbiochem, CN Biosciences UK, Boulevard Industrial Park, Padge Road, Beeston, Nottingham, UK) was covalently immobilised on the surface of a sensor chip (CM5) (from Biacore, SE) by continuous flow of reagents over the sensor chip surface at 5 μl/min. The carboxyl groups on the dextran chip were activated by freshly mixed N-ethyl-N′(dimethylaminopropyl)carbodiimide hydrochloride/N-hydroxysuccinimide (EDC/NHS; 1:1 v/v) (from Biacore, SE) for 2 minutes. This was followed by Factor D (100 μg/ml) diluted in sodium maleate, pH 3.1, (20 MM) to 20 μg/ml for 2 minutes. Injection of factor D was repeated until steady state RU readings were obtained, after which excess activated carboxyl groups were capped with ethanolamine hydrochloride pH 8.5 (EA-HCl)(1 M). Immobilised protein was treated with regeneration buffer (1M sodium chloride/PBS) to remove non-covalently bound ligand.

Recombinant polypeptide purified as described in example 6 was diluted in PBS pH 7.4 to various concentrations (25, 50, 75, 100, and 250 nM). Concentration-dependent binding of polypeptide to immobilised factor D was measured from resonance sensorgrams obtained on passing the polypeptide over the sensor chip after subtraction of background resonance units obtained from a simultaneously run chip lacking factor D. Concentration-dependent binding of the polypeptide to factor D at 25° C. was confirmed by the difference between the responses before and after the injection.

1. A polypeptide, including an isolated, purified or recombinant polypeptide, having a molecular weight in the range of from 7,000-17,000 Da, as measured by mass spectrometry, which polypeptide is both derivable from ectoparasitic leeches (particularly from leech species of the order Rhynchobdellida and more particularly those of the genus Placobdella, especially of the species Placobdella papillifera), and capable of inhibiting the alternative route of complement activation but which polypeptide has substantially no effect on complement activation by the classical route.

2. A polypeptide according to claim 1, comprising the following general formula [SEQ ID NO: 50] in which amino acids are represented by their conventional single letter codes:
X1-E-F-Q-D-X2-K-K-S-S-D-X3-E-T-L-E-L-R-X4-N-K-X5 [SEQ ID NO: 50]
wherein:
X1 is a hydrogen atom (H) or any naturally-occurring amino acid or sequence of amino acids;
X2 is any single amino acid;
X3 is any single amino acid;
X4 is any single amino acid;
X5 is an amino acid sequence comprising naturally-occurring amino acids, one or more of which may comprise post-translational modifications, such as glycosylation at asparagine, serine or threonine; and/or sulphato- or phospho- groups on tyrosine.

3. A polypeptide according to claim 2 wherein X1 is valine, which is a polypeptide of [SEQ ID NO: 50] in which the first 21 amino acids from the N-terminus of the mature polypeptide comprise [SEQ ID NO: 1]:
V-E-F-Q-D-X-K-K-S-S-D-X-E-T-L-E-L-R-X-N-K- [SEQ ID NO:1]
wherein each X represents a single amino acid, which may be the same or different

4. A polypeptide according to claim 2 or claim 3, wherein one or more of X, X2, X3 and/or X4 is/are cysteine.

5. A polypeptide according to any preceding claim comprising [SEQ ID NO: 50], wherein X5 is the amino acid sequence [SEQ ID NO: 51]:
-N-T-S-K-C-E-C-R-N-Q-V-C-P-R-A-C-P-D-G-K-Y-K-L-D-E-Y-G-C-K-R-C-L-C-X6 [SEQID NO:51]
wherein X6 is an amino acid sequence comprising naturally occurring amino acids, one or more of which may comprise post-translational modifications, such as glycosylation at asparagine, serine or threonine; and/or sulphato- or phospho- groups on tyrosine.

6. A polypeptide according to claim 5, wherein X6 is an amino acid sequence selected from one of the following [SEQ ID NOS: 54 and 21 to 23]:
-Q-G-C-N-E-A-Q-C-R [SEQ ID NO: 54];
-Q-G-C-N-E-A-Q-C-R-K-L-C-W-Y-G-F-T-T-D-E-N-G-C-E-S-Y-C-K-C-N-T-K-E-T-A-C-K-N-V-L-C-S-D-S-Y-Q-C-D-P-E-S-G-N-C-V-A-V-T-P-G-K-E-H-D-Y-Y-S-Y-N-D-D-D-D-E-D-K [SEQ ID NO:21];
-Q-G-C-N-E-A-Q-C-R-K-L-C-W-Y-G-F-T-T-D-E-N-G-C-E-S-Y-C-K-C-N-T-K-E-T-A-C-K-N-V-L-C-S-D-S-Y-Q-C-D-P-E-S-G-N-C-V-A-V-I-P-G-K-E-H-D-Y-Y-S-Y-N-D-D-D-D-E-D-K [SEQ ID NO: 22];
and
-Q-G-C-N-E-A-Q-C-R-K-L-C-W-Y-G-F-T-T-D-E-N-G-C-E-S-Y-C-K-C-N-T-K-E-T-A-C-K-N-V-L-C-S-E-S-Y-Q-C-D-P-E-S-G-N-C-V-A-V-I-P-G-K-E-H-D-Y-Y-S-Y-N-D-D-D-E-D-K [SEQ ID NO: 23].

7. A polypeptide according to any preceding claim having the following general formula [SEQ ID.NO: 60]:
X7-K-L-C-W-Y-G-F-T-T-D-E-N-G-C-E-S-Y-C-K-C-N-T-K-E-T-A-C-K-N-V-L-C-S-X8-S-Y-Q-C-D-P-E-S-G-N-C-V-A-V-X9-P-G-K-E-H-D-Y-Y-S-Y-N-D-D-D-X10 [SEQ ID NO: 60]
wherein:
X7 is either a hydrogen atom or an amino acid sequence comprising naturally-occurring amino acids, one or more of which may have post-translational modifications such as glycosylation at asparagine, serine or threonine;
X8 is D or E;
X9 is T or I; and
X10 is -D-E-D-K or -E-D-K

8. A polypeptide according to claim 7, wherein X7 is bonded to a peptide in which:
X8 is D, X9 is T and X10 is -D-E-D-K, as in (SEQ ID NO: 30]:
-K-L-C-W-Y-G-F-T-T-D-E-N-G-C-E-S-Y-C-K-C-N-T-K-E-T-A-C-K-N-V-L-C-S-D-S-Y-Q-C-D-P-E-S-G-N-C-V-A-V-T-P-G-K-E-H-D-Y-Y-S-Y-N-D-D-D-D-E-D-K [SEQ ID NO: 30]
or
X8 is D, X9 is 1 and X10 is -D-E-D-K, as in [SEQ ID NO:31]
-K-L-C-W-Y-G-F-T-T-D-E-N-G-C-E-S-Y-C-K-C-N-T-K-E-T-A-C-K-N-V-L-C-S-D-S-Y-Q-C-D-P-E-S-G-N-C-V-A-V-I-P-G-K-E-H-D-Y-Y-S-Y-N-D-D-D-D-E-D-K [SEQ ID NO: 31]
or
X8 is E, X9 is 1 and X10 is -E-D-K, as in [SEQ ID NO: 32]:
-K-L-C-W-Y-G-F-T-T-D-E-N-G-C-E-S-Y-C-K-C-N-T-K-E-T-A-C-K-N-V-L-C-S-E-S-Y-Q-C-D-P-E-S-G-N-C-V-A-V-I-P-G-K-E-H-D-Y-Y-S-Y-N-D-D-D-E-D-K [SEQ ID NO: 32].

9. A polypeptide according to claim 7 or claim 8, wherein X7 is the sequence [SEQ ID NO: 61]:
X11-C-N-E-A-Q-C-R- [SEQ ID NO: 61]
wherein X11 is selected from G; Q-G; and [SEQ ID NO: 62]:
X12-E-C-R-N-Q-V-C-P-R-A-C-P-D-G-K-Y-K-L-D-E-Y-G-C-K-R-C-L-C-Q-G- [SEQ ID NO:62]
wherein X12 is either a hydrogen atom or [SEQ ID NO: 63]:
X13-N-T-S-K-C- [SEQ ID NO: 63],
wherein X13 is a sequence of [SEQ ID NO: 50].

10. A polypeptide according to any preceding claim comprising any one of [SEQ ID NOS: 15 to 17]:
V-E-F-Q-D-C-K-K-S-S-D-C-E-T-L-E-L-R-C-N-K-N-T-S-K-C-E-C-R-N-Q-V-C-P-R-A-C-P-D-G-K-Y-K-L-D-E-Y-G-C-K-R-C-L-C-Q-G-C-N-E-A-Q-C-R-K-L-C-W-Y-G-F-T-T-D-E-N-G-C-E-S-Y-C-K-C-N-T-K-E-T-A-C-K-N-V-L-C-S-D-S-Y-Q-C-D-P-E-S-G-N-C-V-A-V-T-P-G-K-E-H-D-Y-Y-S-Y-N-D-D-D-D-E-D-K [SEQ ID NO: 15];
V-E-F-Q-D-C-K-K-S-S-D-C-E-T-L-E-L-R-C-N-K-N-T-S-K-C-E-C-R-N-Q-V-C-P-R-A-C-P-D-G-K-Y-K-L-D-E-Y-G-C-K-R-C-L-C-Q-G-C-N-E-A-Q-C-R-K-L-C-W-Y-G-F-T-T-D-E-N-G-C-E-S-Y-C-K-C-N-T-K-E-T-A-C-K-N-V-L-C-S-D-S-Y-Q-C-D-P-E-S-G-N-C-V-A-V-I-P-G-K-E-H-D-Y-Y-S-Y-N-D-D-D-D-E-D-K [SEQ ID NO: 16];
and
V-E-F-Q-D-C-K-K-S-S-D-C-E-T-L-E-L-R-C-N-K-N-T-S-K-C-E-C-R-N-Q-V-C-P-R-A-C-P-D-G-K-Y-K-L-D-E-Y-G-C-K-R-C-L-C-Q-G-C-N-E-A-Q-C-R-K-L-C-W-Y-G-F-T-T-D-E-N-G-C-E-S-Y-C-K-C-N-T-K-E-T-A-C-K-N-V-L-C-S-E-S-Y-Q-C-D-P-E-S-G-N-C-V-A-V-I-P-G-K-E-H-D-Y-Y-S-Y-N-D-D-D-E-D-K [SEQ ID NO: 17]

11. A polypeptide according to claim 1, which is a derivative of a polypeptide according to any of claims 2 to 10 having similar or substantially the same biological activity thereas.

12. A polypeptide according to claim 11, wherein the derivative is selected from: bioprecursors, including sequences according to any preceding claim further comprising a leader or signal sequence; modifications thereof, including sequences glycosylated or sulphated by post-translational processes or by the formation of disulphide bonds between cysteine residues; disulphide-linked double-chained homologues; and sequences in which one or more amino acid(s) is/are varied, including polymorphisms; isoforms; truncated and extended forms; and salts of any of the foregoing.

13. A nucleic acid sequence, including an isolated, purified or recombinant nucleic acid sequence, comprising:
(a) a nucleic acid sequence encoding a polypeptide according to any preceding claim;
(b) a sequence substantially homologous to or that hybridises to sequence (a) under stringent conditions;
(c) a sequence substantially homologous to or that hybridises to the sequences (a) or (b) but for degeneracy of the genetic code; and
(d) an oligonucleotide specific for any of the sequences (a), (b) or (c) above.

14. A nucleic acid sequence, including an isolated, purified or recombinant nucleic acid sequence, which nucleic acid sequence has at least 90% identity of its nucleotide bases with those of the sequence according to claim 13 (a), in matching positions in the sequence, provided that up to six bases may be omitted or added therein and provided that such homologous sequences encode a polypeptide having similar or substantially the same biological activity as the polypeptides according to any of claims 1 to 12.

15. An oligonucleotide specific for any of the nucleic acid sequences according to claim 13 or claim 14, which oligonucleotide comprises a sequence selected from [SEQ ID NOS: 33 to 37]:
GC(CT) TC(AG) TT(AG) CA(AGCT) CC(CT) TG(AG) CA [SEQ ID NO: 33];
GGG GTC GGT AGT TTT GGC GGT AGA G [SEQ ID NO: 34];
CGG GCA GGT ATC ATA ATG [SEQ ID NO: 35];
AGT CGT TCG TTC GTT TTC [SEQ ID NO: 36];
and
ACT GCA GAG TCG TTC GTT CGT TTT CAT TTA TC [SEQ ID NO: 37].

16. A nucleic acid sequence according to any of claims 13 to 15, wherein the sequence is a DNA or RNA sequence, including cDNA or mRNA.

17. A DNA sequence according to any of claims 13 to 16, which sequence comprises any of [SEQ ID NOS: 4 to 5].

18. A DNA sequence according to any of claims 13 to 16, which sequence comprises any of [SEQ ID NOS: 6 to 9 and 13].

19. A method for the preparation of a polypeptide according to any of claims 1 to 12, which method comprises:
(a) isolation and/or purification from material derivable from Rhynchobdellida leeches; or
(b) expression of a nucleic acid sequence encoding the polypeptide and, optionally, isolation and/or purification of the resulting polypeptide.

20. A recombinant construct comprising any nucleic acid sequence according to any of claims 13 to 18.

21. A vector comprising a construct according to claim 20.

22. A host transformed or transfected by a vector according to claim 21.

23. A cell, cell line, plasmid, virus, live organism or other vehicle that has been genetically- or protein-engineered to produce a polypeptide according to any of claims 1 to 12, said cell, cell line, plasmid, virus, live organism or other vehicle having incorporated expressably therein a sequence according to any of claims 13 to 18.

24. A cell or cell line according to claim 23 for use in therapy.

25. A pharmaceutical formulation comprising a polypeptide according to any of claims 1 to 12 and a pharmaceutically acceptable carrier therefor.

26. The use of a polypeptide according to any of claims 1 to 12 or a nucleic acid sequence coding for the polypeptide in medicine, including protein or gene therapy.

27. The use of a polypeptide according to any of claims 1 to 12 in the manufacture of a medicament.

28. The use of a polypeptide according to any of claims 1 to 12 to inhibit one or more steps in the alternative pathway of complement activation.

29. The use of a polypeptide according to any of claims 1 to 12 to interact with complement factor D and/or the C3bBb complex.

30. The use of a polypeptide according to any of claims 1 to 12 to selectively inhibit the alternative pathway of complement activation, compared to its inhibition of the classical and/or coagulation (blood clotting) pathways.

31. A method for the treatment or prevention of a condition in a patient, which condition involves activation of the alternative complement pathway, which method comprises administration to said patient of a non-toxic, inhibitory amount of a polypeptide according to any of claims 1 to 12.

32. A use or method according to any of claims 26 to 31 for a condition selected from: haemodialysis and cardiopulmonary bypass; the presence of in-dwelling catheters and intra-arterial stents; rejection of transplanted organs or tissues; auto-immune diseases including lupus arthritis; rheumatoid arthritis; glomerulonephritis; nephritis; nephropathy; sepsis; injury caused to tissues by reperfusion after an ischaemic period and other conditions associated with activation of complement, including anaphylaxis, asthma, skin reactions, infections, sickle cell anaemia and haemolytic anaemia.

33. A polypeptide, nucleic acid sequence, method construct vector, host, cell line, formulation or use substantially as hereinbefore described, with particular reference to the Examples.
